(19) United States
(12) Patent Application Publication
(10) Pub. No.: US 2022/0354962 A1
(43) Pub. Date: Nov. 10, 2022

(54) RAPID PRODUCTION OF BISPECIFIC ANTIBODIES FROM OFF-THE-SHELF IGGS WITH HIGH YIELD AND PURITY

(71) Applicant: THE TRUSTEES OF THE UNIVERSITY OF PENNSYLVANIA, Philadelphia, PA (US)

(72) Inventors: Andrew TSOURKAS, Bryn Mawr, PA (US); Linghan Mei, Urumqi (CN); Fabiana Zappala, Philadelphia, PA (US)

(73) Assignee: THE TRUSTEES OF THE UNIVERSITY OF PENNSYLVANIA, Philadelphia, PA (US)

(21) Appl. No.: 17/533,071

(22) Filed: Nov. 22, 2021

Related U.S. Application Data
(63) Continuation-in-part of application No. 17/411,815, filed on Aug. 25, 2021, now abandoned.
Application No. 17/411,815 is a continuation of application No. 15/573,757, filed on Nov. 13, 2017, now Pat. No. 11,123,440.
(60) Provisional application No. 62/160,130, filed on May 12, 2015.

(51) Int. Cl. A61K 47/68; C07K 16/28; C07K 16/46; C12N 15/62; C07K 14/315; C12N 9/48

(52) U.S. Cl. CPC A61K 47/6889; C07K 16/2803; C07K 16/2809; C07K 16/2887; C07K 16/468; C12N 15/62; A61K 47/6851; C07K 14/315; C12N 9/48; C12Y 304/2207; C07K 2317/31; C07K 2317/622; C07K 2319/70; C07K 2317/52; A61K 2039/572

(57) ABSTRACT

The invention relates to antibody conjugates (e.g., a bispecific antibody), drug and nanoparticle compositions and methods and compositions for generating them. This invention further relates to methods of using these compositions to image, diagnose or treat a disease.

### CROSS-REFERENCE TO RELATED APPLICATIONS

This application is a Continuation-in-Part of U.S. patent application Ser. No. 17/411,815, filed Aug. 25, 2021, which is a Continuation of U.S. patent application Ser. No. 15/573,757 (now U.S. Pat. No. 11,123,440), filed Nov. 13, 2017, which is a National Phase of International Application PCT/US2016/032221, filed May 12, 2016, which claims priority to and the benefit of U.S. Provisional Patent Application 62/160,130, filed May 12, 2015, each of which is incorporated by reference herein in its entirety.

### GOVERNMENT INTEREST STATEMENT

This invention was made with government support under CA241661 and CA187657 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 30, 2022, is named P-609902-US_SL.txt and is 20,384 bytes in size.

### FIELD OF THE INVENTION

The invention relates to antibody conjugates (e.g., a bispecific antibody), drug and nanoparticle compositions and methods and compositions for generating them. This invention further relates to methods of using these compositions for imaging, diagnosing or treating a disease, such as cancer.

### BACKGROUND OF THE INVENTION

Bispecific antibodies have emerged as a promising cancer treatment, with a growing list of encouraging clinical results. For example, blinatumomab, a murine anti-human CD3×anti-human CD19 bispecific antibody has produced clinical remission in precursor B cell acute lymphoblastic leukemia (B-ALL) patients at thousands times lower dosage than conventional antibody therapies, such as rituximab (anti-human CD20). These findings have spurred a great deal of interest and growth in the field, with particular attention being focused on developing new methodologies to generate bispecific antibodies (e.g., Triomabs, BiTEs, Dock and Lock, etc.) in high yields and purity.

Despite continual progress, current bispecific antibody technologies still require a tremendous amount of antibody engineering and cloning up front to generate even a single functional product, which can be time consuming and challenging. Technologies utilizing scFv's (single chain variable fragments) are also faced with concerns over functionality, solubility, stability, avidity, and pharmacokinetics. Adding to the challenges in producing bispecific antibodies is an incomplete understanding of their modes of action. For example, not all anti-CD3 antibodies work equally well to trigger T-cell activation. Given these uncertainties and the high cost and time required for production, a methodology that allows bispecific antibodies to be rapidly produced without the need for antibody engineering and cloning would be cost-effective, significantly increase throughput, and ultimately lead to a deeper understanding of the underlying biological mechanisms that lead to improved therapeutic efficacy.

Accordingly, there exists a need for improved compositions and methods for making bispecific antibodies and other complex antibody formats.

### SUMMARY OF THE INVENTION

In one aspect, provided herein are conjugate molecules or adapters comprising a first binding pair member and a pair of photoreactive antibody binding domains (pAbBDs) that are adapted to site-specifically bind and photo-crosslink to an immunoglobulin, wherein the first binding pair member and a second binding pair member comprise two moieties that form a heterodimer, and wherein the pair of pAbBDs and the first binding pair member are connected via linkers. In some embodiments, the two moieties that form a heterodimer form a covalently linked heterodimer. In some embodiments, the two moieties that form a heterodimer form a non-covalently linked heterodimer. In some embodiments, the two moieties that form a heterodimer are two protein moieties that form a heterodimer. In some embodiments, the two protein moieties that form a heterodimer form a covalently linked heterodimer. In some embodiments, the two protein moieties that form a heterodimer form a non-covalently linked heterodimer. Also provided herein are nucleic acids and vectors that encode the foregoing adapters, including expression vectors that express the foregoing adapters. Further provided herein are cells that express the foregoing adapters.

In one aspect, provided herein are methods of producing a bispecific antibody from a first antibody or antigen-binding fragment and a second antibody or antigen-binding fragment, the methods comprising the steps of: (a) site-specifically linking the first antibody or antigen-binding fragment to a first adapter comprising a first antibody binding domain (AbBD) attached to or modified with a first binding pair member to form a first adapter-antibody conjugate; (b) site-specifically linking the second antibody or antigen-binding fragment to a second adapter comprising a second antibody binding domain (AbBD) attached to or modified with a second binding pair member to form a second adapter-antibody conjugate; and (c) contacting the first and second adapter-antibody conjugates under conditions where the first and second binding pair members bind to each other to form a bispecific antibody.

In one aspect, provided herein are methods of producing a bispecific antibody from a first antibody or antigen-binding fragment and a second antibody or antigen-binding fragment, the methods comprising the steps of: (a) providing a first adapter comprising a first binding pair member and a first pair of photoreactive antibody binding domains (pAbBDs) that are adapted to site-specifically bind and photo-crosslink to the first antibody or antigen-binding fragment, and wherein the first pair of pAbBDs and the first binding pair member are connected via linkers; (b) providing a second adapter comprising a second binding pair member and a second pair of photoreactive antibody binding domains (pAbBDs) that are adapted to site-specifically bind and photo-crosslink to the second antibody or antigen-binding fragment, wherein each of the second pair of pAbBDs is connected via a linker to the second binding pair member, and wherein the first and second binding pair members comprise two moieties that form a heterodimer; (c) site-specifically photo-crosslinking each of the first pair of pAbBDs to the first antibody or antigen-binding fragment to form a first adapter-antibody conjugate; (d) site-specifically photo-crosslinking each of the second pair of pAbBDs to the second antibody or antigen-binding fragment to form a second adapter-antibody conjugate; and (e) contacting the first and second adapter-antibody conjugates under conditions where the first and second binding pair members bind to each other to form a bispecific antibody. In some embodiments, the method further comprises the step of purifying or isolating the bispecific antibody formed in step (e). In some embodiments, the method further comprises the step of purifying or isolating the first adapter-antibody conjugate after step (c). In some embodiments, the method further comprises the step of purifying or isolating the second adapter-antibody conjugate after step (d). In some embodiments, the two moieties that form a heterodimer form a covalently linked heterodimer. In some embodiments, the two moieties that form a heterodimer form a non-covalently linked heterodimer. In some embodiments, the two moieties that form a heterodimer are two protein moieties that form a heterodimer. In some embodiments, the two protein moieties that form a heterodimer form a covalently linked heterodimer. In some embodiments, the two protein moieties that form a heterodimer form a non-covalently linked heterodimer.

In another aspect, provided herein are methods of producing a bispecific antibody from a first antibody or antigen-binding fragment and a second antibody or antigen-binding fragment, the method comprising the steps of: (a) providing a first adapter comprising a first binding pair member and a first pair of photoreactive antibody binding domains (pAbBDs) that are adapted to site-specifically bind and photo-crosslink to the first antibody or antigen-binding fragment, and wherein the first pair of pAbBDs and the first binding pair member are connected via linkers; (b) providing a second adapter comprising a second binding pair member and a second pair of photoreactive antibody binding domains (pAbBDs) that are adapted to site-specifically bind and photo-crosslink to the second antibody or antigen-binding fragment, wherein each of the second pair of pAbBDs is connected via a linker to the second binding pair member, and wherein the first and second binding pair members comprise two moieties that form a heterodimer; (c) site-specifically photo-crosslinking each of the first pair of pAbBDs to the first antibody or antigen-binding fragment to form a first adapter-antibody conjugate; (d) contacting the first adapter-antibody conjugate with the second adapter under conditions where the first and second binding pair members bind to each other; and (e) site-specifically photo-crosslinking each of the second pair of pAbBDs to the second antibody or antigen-binding fragment to the second adapter to form a bispecific antibody.

In another aspect, provided herein are methods of producing a bispecific antibody from a first antibody or antigen-binding fragment and a second antibody or antigen-binding fragment, the method comprising the steps of: (a) providing a first adapter comprising a first binding pair member and a first pair of photoreactive antibody binding domains (pAbBDs) that are adapted to site-specifically bind and photo-crosslink to the first antibody or antigen-binding fragment, and wherein the first pair of pAbBDs and the first binding pair member are connected via linkers; (b) providing a second adapter comprising a second binding pair member and a second pair of photoreactive antibody binding domains (pAbBDs) that are adapted to site-specifically bind and photo-crosslink to the second antibody or antigen-binding fragment, wherein each of the second pair of pAbBDs is connected via a linker to the second binding pair member, and wherein the first and second binding pair members comprise two moieties that form a heterodimer; (c) contacting the first adapter comprising and the second adapter under conditions where the first and second binding pair members bind to each other; (d) site-specifically photo-crosslinking each of the first pair of pAbBDs to the first antibody or antigen-binding fragment to the first adapter; and (e) site-specifically photo-crosslinking the second antibody or antigen-binding fragment to the second adapter to form a bispecific antibody.

Other features and advantages of this invention will become apparent from the following detailed description examples and figures. It should be understood, however, that the detailed description and specific examples while indicating preferred embodiments are given by way of illustration only, since various changes and modifications within the spirit and scope of the invention will become apparent to those skilled in the art.

### BRIEF DESCRIPTION OF THE DRAWINGS

The following drawings form part of this specification and are included to further demonstrate certain aspects of the disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1C. Schematic of one method for the production of bispecific antibodies. (FIG. 1A) Protein Z is produced in an entirely recombinant manner. This is achieved by using E. coli that have been engineered to incorporate the unnatural amino acid, BPA, into proteins during translation. A sortase-mediated Expressed Protein Ligation (EPL) technique can be used to ligate peptides to the carboxy-terminus of recombinant proteins during the affinity purification process. This sortase-mediated EPL technique is described in greater detail in PCT Appl. PCT/US2014/030208 (filed Mar. 17, 2014), which is hereby incorporated in its entirety. A crosslinking group (azide, alkyne, biotin, maleimide, etc.) is included on this peptide. FIG. 1A discloses SEQ ID NOS 21, 23, 21, and 26, respectively, in order of appearance. (FIG. 1B) Protein Z-conjugates can be photocrosslinked to each IgG. (FIG. 1C) Azide-modified IgG-Protein Z-peptide conjugates can be efficiently conjugated to ADIBO-modified IgG-Protein Z-peptide conjugates to form bispecific antibodies.

FIGS. 2A-2C. (FIG. 2A) Plot of STEPL-ligation efficiency and the % of the glycine peptide (GGG) utilized, relative to the amount of expressed protein Z (Pz)-SrtA on the affinity column. (FIG. 2B) SDS-PAGE of unmodified Pz and Pz-conjugate. Here, Pz is ligated to a peptide labeled with a fluorophore and azide. (FIG. 2C) Fluorescent image of the gel.

FIGS. 3A-3B. Protein Z and Rituximab (Ritux) samples with and without UV crosslinking were run on a (FIG. 3A) reducing SDS-PAGE gel and a (FIG. 3B) non-reducing gel.

FIG. 4. Reducing SDS-PAGE of Protein Z and Rituximab (Ritux) with and without UV crosslinking.

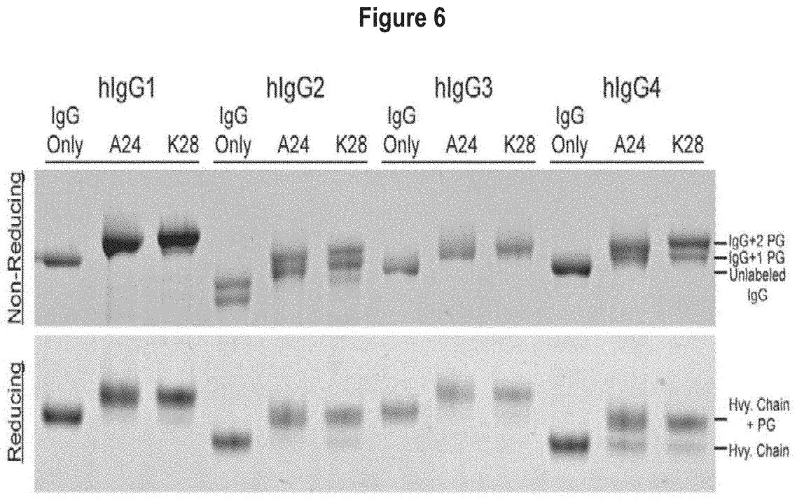

FIG. 5. Illustration of IgG being photocrosslinked with a Protein G-based adapter protein. The Protein G adapter (blue) contains a customizable conjugate at its C-terminus and the unnatural amino acid benzoylphenylalanine (BPA), whose UV-active benzophenone side chain is shown in red, in the Fc binding domain. When bound to the Fc region of IgG and activated by long wavelength UV light (365 nm), a covalent bond is formed between Protein G and IgG. Either one or two Protein Gs can be conjugated onto each Fc (second one is shown faded).

FIG. 6. Non-reducing and reducing SDS-PAGE gels of various human IgG subclasses alone or after photocrosslinking with Protein G-based adapter proteins. The adapter proteins possessed either an A24BPA or K28BPA substitution. Conjugation was done for one hour and 30 minutes using four equivalents of Protein G.

FIGS. 7A-7C. Kinetics and efficiency of IgG-adapter protein crosslinking. Non-reducing and reducing SDS-PAGE of cetuximab (Cetux, human IgG1) alone or after photocrosslinking with Protein G (PG)-based adapter proteins. The adapter proteins possessed either an (FIG. 7A) A24BPA or (FIG. 7B) K28BPA substitution. UV crosslinking was performed for varying periods of time using four equivalents of the adapter proteins. Image analysis of non-reducing gels are shown on the right. (FIG. 7C) UV crosslinking was performed for one hour and 30 minutes with various molar ratios of adapter protein-to-IgG. The adapter proteins possessed either an A24BPA or K28BPA substitution. Non-reducing and reducing SDS-PAGE gels of cetuximab alone or after photocrosslinking with the Protein G-based adapter proteins are shown. Image analysis of non-reducing gels are shown on the right.

FIG. 8. Antibody binding affinity with and without LASIC. Unmodified Cetuximab (Human IgG1 anti-human EGFR antibodies), or Cetuximab that was subject to LASIC using the A24BPA Protein G adapter protein, were applied to EGFR positive KB cells. The extent of cell labeling was quantified by flow cytometry using a fluorescent anti-human secondary antibody.

FIG. 9. Effect of impure IgG samples on LASIC. 0.25 μg of Human IgG2, either alone (lanes 3, 5) or with 25 μg BSA (lanes 2,4), were conjugated with a TAMRA labeled Protein G adapter protein (lane 1). Samples were run on an SDS-PAGE reducing gel and white light and fluorescence images of the gel were acquired.

FIGS. 10A-10C. Modification of IgG with various functional moieties using Protein G adapters. Protein G adapters made with peptides containing either (FIG. 10A) TAMRA-DBCO (lane 3), (FIG. 10B) FAM-Azide (lane 7), or (FIG. 10C) Biotin (lane 11), were LASIC conjugated onto human IgG1 (Cetux, lanes 2 and 6) or mouse IgG2a (OKT3, lane 10). Unconjugated IgGs are shown in lanes 1 (Cetux), 5 (Cetux), and 9 (OKT3). Conjugates remained active as demonstrated by click reactions (Lane 4: Click with Peg-Azide; Lane 8: Click with PG-TAMRA-DBCO) or by Western blot with Streptavind-IRdye800 (lane 10). The arrow (>) indicates Protein G-labeled heavy chains; The asterisk (*) indicates click product).

FIGS. 11A-11C. Controlled labelling of IgG with one or two Protein G adapters. (FIG. 11A) Schematic of IgG being labelled with a single Protein G adapter (i.e. mono-conjugated product) by first preadsorbing IgG onto Protein A or G resins, leaving only one heavy chain available for conjugation. (FIG. 11B) Schematic describing the purification of mono-conjugated product by capturing it from product mixtures of mono- and di-conjugated products. Di-conjugated IgGs cannot bind to Protein A or G resin. (FIG. 11C) Non-reducing SDS-PAGE gel showing mono-conjugated Cetuximab using the method described in (FIG. 11B).

FIG. 12. Protein G with selected side chains depicted.

FIGS. 13A-13B. Screening Protein G adapters for ability to label (FIG. 13A) human and (FIG. 13B) mice IgGs. Reducing SDS-PAGE gel showing that human IgG1 (cetuximab) can be specifically conjugated on the heavy chain by several different Protein G adapters. A24Bpa and K28Bpa showed the best conjugation efficiencies.

FIGS. 14A-14C. (FIG. 14A) Model of Protein G binding to Fc (1FCC), and (FIG. 14B-14C) IgG sequence alignment. FIGS. 14B-14C discloses SEQ ID NOS 3-16, 27, and 27-39, respectively, in order of appearance.

FIG. 15. Storage of Protein G in room temperature (RT) and under ambient lighting (AL) does not affect its ability to label IgG.

FIG. 16. Native IgGs (cetuximab and OKT3) were site-specifically modified on their heavy chains using either SpyTag (Mod.A) or SpyCatcher (Mod.B). SpyCatcher reacts specifically with SpyTag, to give bispecific IgG dimers that are entirely composed of two different IgG (cetuximab×OKT3).

FIG. 17. Bispecificity Confirmed by Western. Western blotting confirms heterodimer formation. A non-reducing SDS-PAGE was probed first with anti-mouse IRdye800 2′ (labeling OKT3). The blot was then stripped, followed by anti-human IRdye800 2′ (labeling Cetux).

FIG. 18. Kinetics of bispecific antibody formation. Dimer formation is efficient and stoichiometric. Yield is >50%, of total inputting IgGs. It is reproducible at 0.5-1 mg scale. Modification of individual IgG with either Mod.A (SpyTag) or Mod.B (SpyCatcher) takes 60-120 min. Bispecific antibody formation is fast. Nearly plateaus after 30 minutes. No increase in multimer formation seen over time, which is likely due to unfavorable sterics effects.

FIG. 19. Easy purification of bispecific antibody dimers from monomers and multimers.

FIG. 20. Modular bispecific antibodies made using azide-DBCO click chemistry. Protein Z-IgG was reacted with a second Protein Z (representative second “targeting ligand”).

FIG. 21. Modular bispecific antibodies made using Tetrazine-TCO click chemistry. A bispecific consisting of OKT3 and Rituximab is shown.

FIG. 22. A Protein Z-Protein G fusion protein with orthoganol specificity for a particular IgG subtype was made and used to make a bispecific antibody. Protein Z^{L17} (Pz^{L17}) conjugates mIgG2a (OKT3) only. No hIgG1 (Cetux) conjugation is observed except at very high Protein Z^{L17} concentrations. Protein G^{K28}(PG^{K28}) conjugates hIgG1 (Cetux) only. No mIgG2a (OKT3) conjugation is observed except at very high Protein G^{K28} concentrations. A fusion protein of Protein Z^{L17}-Protein G^{K28} is hence “Orthogonal” and conjugates OKT3 via Protein Z^{L17} and Cetux via Protein G^{K28} to create the desired heterodimers between Cetux and OKT3.

FIG. 23. Pz^{L17}-PG^{K28} Orthogonal bispecific antibodies. Samples from FIG. 22 were analyzed via a non-reducing gel. Even at the highest Protein Pz^{L17}-PG^{K28} concentration (Lane 4 and 5; 12 μL) only scant homodimers formed, which suggests dimers in Lanes 1-3 are mostly heterodimers.

FIG. 24. Schematic describing method for rapid production of bispecific antibodies. An anti-CD3 scFv is fused to a photoreactive antibody-binding domain (AbBD). Administration of non-damaging long-wavelength UV light allows for covalent attachment of the fusion protein to the Fc-region of IgG.

FIG. 25. Schematic of mono-conjugated and di-conjugated bispecific antibodies.

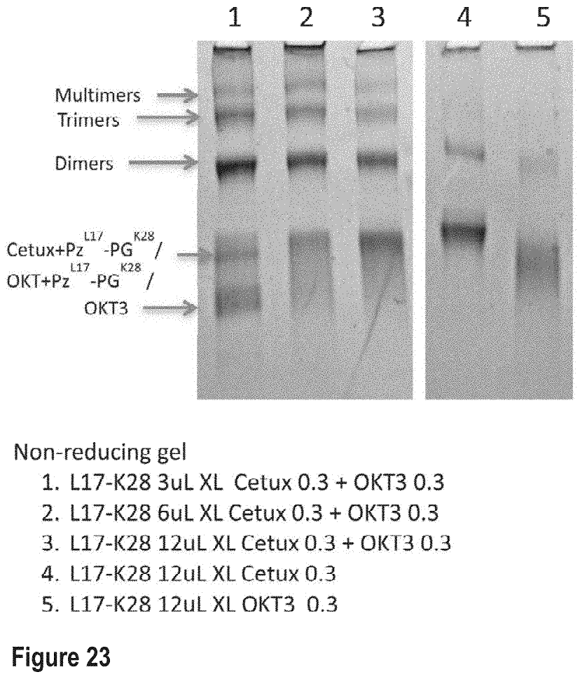

FIG. 26. Schematic describing the production of a photoreactive AbBD-scFv fusion protein with a c-terminal-modification (red star) that was introduced using sortase-tag expressed protein ligation (STEPL). FIG. 26 discloses SEQ ID NOS 21, 23, 21, 40, and 41, respectively, in order of appearance.

FIG. 27. Reducing SDS-PAGE of four different human antibodies—Rituximab, Cetuximab, Trastuzumab, and IgG4—alone or after photo-crosslinking with AbBD-anti-CD3 scFv. Free AbBD-scFv was efficiently removed via filtration.

FIG. 28. T cell-mediated cell lysis assay. Di-conjugated rituximab×anti-CD3 scFv bispecific antibodies were incubated with CD20-positive Jeko B cells (▴) or CD20-negative K562 cells (◯) for 24 hrs in the presence of PBMCs at an effector-to-target ratio of 10:1. Analogous studies were performed with rituximab alone (▪) or rituximab+anti-CD3 scFv (X) with Jeko B cells in the presence of PBMCs at an effector-to-target ratio of 10:1. All data points are mean±SD of triplicate wells.

FIGS. 29A-29B. (FIG. 29A) Plot of STEPL ligation efficiency and the % of triglycine peptide utilized, relative to the amount of expressed AbBD-SrtA. The horizontal dashed line represents 95% purity of conjugated product. (FIG. 29B) SDS-PAGE of unmodified AbBD and AbBD ligated to a peptide with an azide and fluorophore (AzFP). White light and fluorescent images of gel are shown.

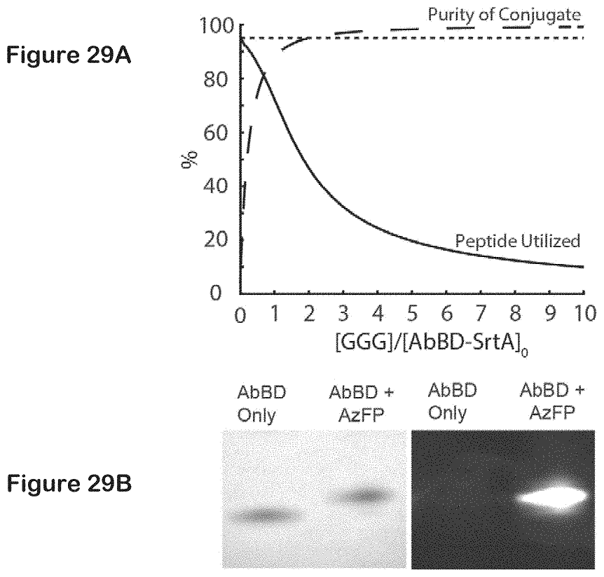

FIG. 30. TLC of di-conjugated IgG-NOTA after labeling with various amounts of ⁶⁴Cu.

FIGS. 31A-31B. Schematic representation of the two different schemes used to rapidly produce bispecific antibodies. (FIG. 31A) In scheme 1, each component IgG is first conjugated to either a photoreactive antibody-binding domain fused to SpyCatcher (pAbBD-SC) or SpyTag (pAbBD-ST). Photocrosslinking is performed by exposing the sample to non-damaging 365 nm light for 2 hours. The IgG-SC and IgG-ST conjugates are then combined to form a BsAb via SpyCatcher-SpyTag isopeptide bond formation. (FIG. 31B) In scheme 2, in one alternative, each component IgG is conjugated to either pAbBD-SC-pAbBD or pAbBD-ST-pAbBD. Using a construct with two pAbBDs flanking SC or ST, the IgG is more likely to be labeled with only a single SpyCatcher or SpyTag. Once IgGs are labeled with SpyCatcher or SpyTag, their conjugates are combined to form a BsAb. In scheme 2, in a second alternative, each component IgG is conjugated to either pAbBD-pAbBD-SC or pAbBD-pAbBD-ST. Using a construct with two pAbBDs with a linker between, the IgG is more likely to be labeled with only a single SpyCatcher or SpyTag, which are placed at the N- or C-terminus. Once IgGs are labeled with SpyCatcher or SpyTag, their conjugates are combined to form a BsAb.

FIGS. 32A-32F. SDS-PAGE gels of precursor IgG conjugates and BsAbs produced via the scheme 1 conjugation method. (FIG. 32A) SDS-PAGE of Trastuzumab (Tras) before and after photocrosslinking to pAbBD-SC, under reducing (R) and non-reducing (NR) conditions. (FIG. 32B) Protein G resin was used to capture and enrich for IgG conjugated with one or no pAbBD-SC fusion proteins. The fraction of the photocrosslinked sample that was captured by the Protein G resin as well as the flow-thru (i.e. uncaptured) was evaluated by SDS-PAGE. (FIG. 32C) SDS-PAGE of Cetuximab (Cetux) before and after photocrosslinking to pAbBD-ST. (FIG. 32D) SDS-PAGE of the photocrosslinked IgG-ST after capture and enrichment by Protein G resin, as well as the flow-thru (i.e. uncaptured). Photocrosslinking reactions between IgG and pAbBD-SC or pAbBD-ST were performed using non-damaging 365 nm light, for 2 hours, at a pAbBD to IgG ratio of 1.2:1. (FIG. 32E) Tras-SC and Cetux-ST were mixed at an equimolar ratio, to generate a Tras×Cetux BsAb, and analyzed by SDS-PAGE before and after purification by size exclusion chromatography (SEC). (FIG. 32F) Cetux-SC and OKT3-ST were mixed at an equimolar ratio, to generate a Cetux×OKT3 BsAb, and analyzed by SDS-PAGE before and after purification by SEC. All gels were stained using SimplyBlue Safe Stain. Protein sizes were confirmed against Novex Sharp Prestained Protein Standard. Each crosslinking experiment was performed on at least three separate occasions. Gel images and quantification are from a representative experiment.

FIGS. 33A-33H. SDS-PAGE gels of IgG photocrosslinked with pAbBD-SC/ST-pAbBD at varying SC/ST-to-IgG ratios and IgG concentrations. Samples containing 0.15 mg/mL IgG (Cetuximab) and varying amounts of (FIG. 33A) pAbBD-SC-pAbBD or (FIG. 33B) pAbBD-ST-pAbBD were exposed to 365 nm light for 2 hours and analyzed by SDS-PAGE, under non-reducing conditions. The gels were stained using SimplyBlue Safe Stain. The intensity of each band on the gel was quantified in ImageJ and the percentage of each molecular species was plotted as a function of the (FIG. 33C) pAbBD-SC-pAbBD-to-IgG ratio and (FIG. 33D) pAbBD-ST-pAbBD-to-IgG ratio. Samples at a fixed 2:1 (FIG. 33E) pAbBD-SC-pAbBD-to-IgG and (FIG. 33F) pAbBD-ST-pAbBD-to-IgG ratio, but with increasing concentrations of IgG were also analyzed by non-reducing SDS-PAGE, following photocrosslinking. The intensity of each band on the gel was quantified in ImageJ and the percentage of each molecular species was plotted as a function of IgG concentration for both (FIG. 33G) IgG-SC and (FIG. 33H) IgG-ST. Each crosslinking experiment was performed on at least three separate occasions. Gel images and quantification are from a representative experiment.

FIGS. 34A-34C. Purification of IgG conjugated to a single SpyCatcher/SpyTag by SEC. (FIG. 34A) SEC trace of Trastuzumab following photocrosslinking with pAbBD-SC-pAbBD. (FIG. 34B) SEC trace of Cetuximab following photocrosslinking with pAbBD-ST-pAbBD. Photocrosslinking reactions between IgG and pAbBD-SC-pAbBD or pAbBD-ST-pAbBD were performed using non-damaging 365 nm light, for 2 hours, at a pAbBD-to-IgG ratio of 2:1 and an IgG concentration of 0.3 mg/mL. (FIG. 34C) Non-reducing SDS-PAGE of Trastzumab crosslinked to pAbBD-SC-pAbBD and Cetuximab crosslinked to pAbBD-ST-pAbBD before and after SEC. Protein gels were stained using SimplyBlue Safe Stain. Each experiment was performed on at least three separate occasions. Gel images are from a representative experiment.

FIGS. 35A-35B. SEC purification of BsAbs produced via Scheme 2. (FIG. 35A) SEC trace of Trastuzumab×Cetuximab BsAb after mixing an equimolar amount of purified Trastuzumab-pAbBD-SC-pAbBD and Cetuximab-pAbBD-SC-pAbBD conjugates. Samples were mixed overnight at 4° C. (FIG. 35B) Non-reducing SDS-PAGE Trastuzumab×Cetuximab BsAb before and after SEC. The gel was stained with SimplyBlue Safe Stain. A representative gel image is shown. Each experiment was performed on at least three separate occasions.

FIGS. 36A-36D. Functional evaluation of BsAbs produced via Scheme 2. Dose-dependent binding of Trastuzumab (circle), Cetuximab (square), and Trastuzumab×Cetuximab BsAbs (triangle) to (FIG. 36A) Her2-positive T6-17 cells and (FIG. 36B) EGFR-positive MDA-MB-468 cells. Each data point represents a set of duplicated wells. (FIG. 36C) T cell mediated cytolysis of EGFR-positive MDA-MB-468 cells as a function of Cetuximab×OKT3 BsAb concentration (triangle). A mixture of equivalent concentrations of un-crosslinked Cetuximab and OKT3 at 10 nM (circle) was included as a negative control. (FIG. 36D) Kinetics of T-cell mediated cytolysis of MDA-MB-468 cells in increasing concentrations of Cetuximab×OKT3 BsAbs (triangle) or an equimolar mixture of Cetuximab and OKT3 (circle). For both Cetuximab×OKT3 BsAb treatment and the equimolar Cetuximab, OKT3 mixture treatment, each data point represents a set of duplicated wells.

### DETAILED DESCRIPTION OF THE INVENTION

The invention provides antibody conjugates (e.g., bispecific antibodies), drug and nanoparticle compositions and methods and compositions for generating them. This invention further provides methods of using these compositions to image, diagnose or treat a disease, such as cancer.

The inventors of this application have developed facile methods for the efficient production of bispecific antibodies from full-length unmodified IgG, without the need for antibody engineering, cloning, or modifications. The bispecific antibodies can be produced with high purity within just a few hours. Several benefits of working with intact IgG are that they are stable, can be produced in high yield, offer high-avidity bivalent binding, and are expected to maintain Fc-effector functions, including antibody dependent cell-mediated cytoxicity (ADCC) and complement-dependent cytotoxicity (CDC).

All types of antibodies are contemplated herein. In one aspect, provided herein are conjugate molecules or adapters that are adapted to site-specifically bind and crosslink to an antibody. In one aspect, provided herein are methods of producing bispecific antibodies. In another aspect, provided herein are methods to site-specifically label an antibody with a chemical or biological moiety. In another aspect, provided herein are methods to site-specifically attach an antibody onto a surface.

In one aspect, provided herein are conjugate molecules or adapters comprising a first binding pair member and a pair of photoreactive antibody binding domains (pAbBDs) that are adapted to site-specifically bind and photo-crosslink to an immunoglobulin, wherein the first binding pair member and a second binding pair member comprise two moieties that form a heterodimer, and wherein the pair of pAbBDs and the first binding pair member are connected via linkers. In some embodiments, the two moieties that form a heterodimer form a covalently linked heterodimer. In some embodiments, the two moieties that form a heterodimer form a non-covalently linked heterodimer. In some embodiments, the two moieties that form a heterodimer are two protein moieties that form a heterodimer. In some embodiments, the two protein moieties that form a heterodimer form a covalently linked heterodimer. In some embodiments, the two protein moieties that form a heterodimer form a non-covalently linked heterodimer. Also provided herein are nucleic acids and vectors that encode the foregoing adapters, including expression vectors that express the foregoing adapters. Further provided herein are cells that express the foregoing adapters.

In some embodiments, each binding pair member comprises a peptide tag or a protein. In some embodiments, the binding pair members are SpyCatcher and SpyTag. In some embodiments, the binding pair members are SnoopCatcher and SnoopTag. In some embodiments, the binding pair members are one half of a split intein and the other half of the split intein.

In some embodiments, a binding pair comprises two proteins that form a heterodimer. In some embodiments, one of the binding pair members comprises a first dimerization domain and the other binding pair member comprises a second dimerization domain, wherein the two dimerization domains form a heterodimer. In one embodiment, the binding pair members are c-Jun and c-Fos. In another embodiment, the binding pair members comprise a leucine zipper. In still another embodiment, the binding pair members comprise peptide Velcro, i.e., two peptides that are predominantly unfolded in isolation but which, when mixed, associate preferentially to form a stable, parallel, coiled-coil heterodimer, such as a leucine zipper (See O'Shea et al. (1993) Curr. Biol. 3:658-667). In another embodiment, the binding pair members are a portion of a split adhesin domain and the remaining portion. In some embodiments, the binding pair members are S-protein and S-tag. In some embodiments, the binding pair members are Strep-tag or Strep-tag II and Streptavidin or Streptactin. In some embodiments, the binding pair members are calmodulin-binding peptide and Calmodulin. In some embodiments, the binding pair members are a leader peptide and a B1 protein pair from a lasso peptide biosynthesis system.

In some embodiments, the binding pair members comprise two protein or peptide moieties that form a heterodimer selected from the group consisting of SpyCatcher and SpyTag; two complementary halves of a split intein; c-Jun and c-Fos; leucine zippers; split adhesin domains; SnoopCatcher and SnoopTag; S-protein and S-Tag; Streptavidin/Streptactin or variants thereof and Strep-tag or Strep-tag II; calmodulin and calmodulin binding peptide; a leader peptide and a B1 protein pair from a lasso peptide biosynthesis system and a binding pair of a dock-and-lock system. In some embodiments, the binding pair members comprise two moieties that can undergo a click reaction. In some embodiments, the binding pair members comprise a pair of complementary oligonucleotides.

In some embodiments, each of the pair of pAbBDs in the adapter site-specifically bind and photo-crosslink to the two heavy chains of a single immunoglobulin making up the antibody. It will therefore be appreciated that the distance between the two pAbBDs in the adapter should be large enough for each of the pAbBDs to bind to each of their binding sites in the two heavy chains of a single immunoglobulin molecule. Therefore, in adapters containing a pair of pAbBDs the distance between the pAbBDs should generally be at least 35-50 amino acids. Thus, in adapters where the two pAbBDs are connected in tandem, the two pAbBDs will generally be connected by a linker of at least 40-50 amino acids, for example, a linker between 50 and 100 amino acids long.

In some embodiments, the linkers are flexible GS-rich linkers. In some embodiments, the flexible GS-rich linkers are (GGS)n linkers, where n is an integer. In some embodiments, the flexible GS-rich linkers have from 3 to 8 GGS repeats (SEQ ID NO: 42). In some embodiments, the flexible GS-rich linkers have from 4 to 30 GGS repeats (SEQ ID NO: 43). In some embodiments, the flexible GS-rich linkers are (GGGS)n linkers, where n is an integer (SEQ ID NO: 19). In some embodiments, the flexible GS-rich linkers have from 1 to 8 GGS repeats (SEQ ID NO: 44). In some embodiments, the flexible GS-rich linkers have from 3 to 8 GGS repeats (SEQ ID NO: 42). In some embodiments, the flexible GS-rich linkers have from 4 to 25 GGS repeats (SEQ ID NO: 45). In some embodiments, the flexible GS-rich linkers are (GGGGS)n linkers, where n is an integer (SEQ ID NO: 20). In some embodiments, the flexible GS-rich linkers have from 1 to 8 GGS repeats (SEQ ID NO: 44). In some embodiments, the flexible GS-rich linkers have from 3 to 8 GGS repeats (SEQ ID NO: 42). In some embodiments, the flexible GS-rich linkers have from 4 to 20 GGS repeats (SEQ ID NO: 46).

In one aspect, provided herein are methods of producing a bispecific antibody from a first antibody or antigen-binding fragment and a second antibody or antigen-binding fragment, the methods comprising the steps of: (a) site-specifically linking the first antibody or antigen-binding fragment to a first adapter comprising a first antibody binding domain (AbBD) attached to or modified with a first binding pair member to form a first adapter-antibody conjugate; (b) site-specifically linking the second antibody or antigen-binding fragment to a second adapter comprising a second antibody binding domain (AbBD) attached to or modified with a second binding pair member to form a second adapter-antibody conjugate; and (c) contacting the first and second adapter-antibody conjugates under conditions where the first and second binding pair members bind to each other to form a bispecific antibody.

In one aspect, provided herein are methods of producing a bispecific antibody from a first antibody or antigen-binding fragment and a second antibody or antigen-binding fragment, the methods comprising the steps of: (a) providing a first adapter comprising a first binding pair member and a first pair of photoreactive antibody binding domains (pAbBDs) that are adapted to site-specifically bind and photo-crosslink to the first antibody or antigen-binding fragment, and wherein the first pair of pAbBDs and the first binding pair member are connected via linkers; (b) providing a second adapter comprising a second binding pair member and a second pair of photoreactive antibody binding domains (pAbBDs) that are adapted to site-specifically bind and photo-crosslink to the second antibody or antigen-binding fragment, wherein each of the second pair of pAbBDs is connected via a linker to the second binding pair member, and wherein the first and second binding pair members comprise two moieties that form a heterodimer; (c) site-specifically photo-crosslinking each of the first pair of pAbBDs to the first antibody or antigen-binding fragment to form a first adapter-antibody conjugate; (d) site-specifically photo-crosslinking each of the second pair of pAbBDs to the second antibody or antigen-binding fragment to form a second adapter-antibody conjugate; and (e) contacting the first and second adapter-antibody conjugates under conditions where the first and second binding pair members bind to each other to form a bispecific antibody. In some embodiments, the method further comprises the step of purifying or isolating the bispecific antibody formed in step (e). In some embodiments, the method further comprises the step of purifying or isolating the first adapter-antibody conjugate after step (c). In some embodiments, the method further comprises the step of purifying or isolating the second adapter-antibody conjugate after step (d). In some embodiments, the two moieties that form a heterodimer form a covalently linked heterodimer. In some embodiments, the two moieties that form a heterodimer form a non-covalently linked heterodimer. In some embodiments, the two moieties that form a heterodimer are two protein moieties that form a heterodimer. In some embodiments, the two protein moieties that form a heterodimer form a covalently linked heterodimer. In some embodiments, the two protein moieties that form a heterodimer form a non-covalently linked heterodimer.

In some embodiments, the first and second antibodies are IgG molecules. In some embodiments, the IgG molecules are native IgG molecules. In some embodiments, the first and second IgG molecules are different subclasses or from different species. In some embodiments, the first and second IgG molecules are the same subclass. In some embodiments, the first and second IgG molecules are from the same species. In some embodiments, at least one IgG molecule is a human IgG molecule. In some embodiments, both IgG molecules are human IgG molecules.

In some embodiments, each of the pair of pAbBDs in the adapter site-specifically bind and photo-crosslink to the two heavy chains making up the antibody. In some embodiments, the two pAbBDs in the adapter are the same as each other. In some embodiments, the two pAbBDs in the adapter are different from each other.

In another aspect, provided herein is a conjugate composition comprising a protein that comprises an antibody-binding domain (AbBD) operably linked to a photoreactive amino acid, wherein said protein is operably linked to an antibody or a fragment thereof. In an exemplary embodiment, the photoreactive amino acid is benzoylphenylalaine (BPA). In one exemplary embodiment, the antibody-binding domain is a domain of protein G (e.g., HTB1).

In another aspect, provided herein is a method of producing a bispecific antibody from a first antibody or antigen-binding fragment and a second antibody or antigen-binding fragment, the method comprising the steps of (a) site-specifically linking the first antibody or antigen-binding fragment to a first adapter comprising a first antibody binding domain (AbBD) attached to or modified with a first member of a binding pair to form a first adapter-antibody conjugate; (b) site-specifically linking the second antibody or antigen-binding fragment to a second adapter comprising a second antibody binding domain (AbBD) attached to or modified with a second member of the binding pair to form a second adapter-antibody conjugate; and (c) contacting the first and second adapter-antibody conjugates under conditions where the first and second members of the binding pair bind to each other to form a bispecific antibody.

In another aspect, provided herein are methods of producing a bispecific antibody from a first antibody or antigen-binding fragment and a second antibody or antigen-binding fragment, the method comprising the steps of: (a) providing a first adapter comprising a first binding pair member and a first pair of photoreactive antibody binding domains (pAbBDs) that are adapted to site-specifically bind and photo-crosslink to the first antibody or antigen-binding fragment, and wherein the first pair of pAbBDs and the first binding pair member are connected via linkers; (b) providing a second adapter comprising a second binding pair member and a second pair of photoreactive antibody binding domains (pAbBDs) that are adapted to site-specifically bind and photo-crosslink to the second antibody or antigen-binding fragment, wherein each of the second pair of pAbBDs is connected via a linker to the second binding pair member, and wherein the first and second binding pair members comprise two moieties that form a heterodimer; (c) site-specifically photo-crosslinking each of the first pair of pAbBDs to the first antibody or antigen-binding fragment to form a first adapter-antibody conjugate; (d) contacting the first adapter-antibody conjugate with the second adapter under conditions where the first and second binding pair members bind to each other; and (e) site-specifically photo-crosslinking each of the second pair of pAbBDs to the second antibody or antigen-binding fragment to the second adapter to form a bispecific antibody. In some embodiments, the method further comprises the step of purifying or isolating the bispecific antibody formed in step (e). In some embodiments, the method further comprises the step of purifying or isolating the product after step (c). In some embodiments, the method further comprises the step of purifying or isolating the product after step (d). In some embodiments, the two moieties that form a heterodimer form a covalently linked heterodimer. In some embodiments, the two moieties that form a heterodimer form a non-covalently linked heterodimer. In some embodiments, the two moieties that form a heterodimer are two protein moieties that form a heterodimer. In some embodiments, the two protein moieties that form a heterodimer form a covalently linked heterodimer. In some embodiments, the two protein moieties that form a heterodimer form a non-covalently linked heterodimer.

In another aspect, provided herein are methods of producing a bispecific antibody from a first antibody or antigen-binding fragment and a second antibody or antigen-binding fragment, the method comprising the steps of: (a) providing a first adapter comprising a first binding pair member and a first pair of photoreactive antibody binding domains (pAbBDs) that are adapted to site-specifically bind and photo-crosslink to the first antibody or antigen-binding fragment, and wherein the first pair of pAbBDs and the first binding pair member are connected via linkers; (b) providing a second adapter comprising a second binding pair member and a second pair of photoreactive antibody binding domains (pAbBDs) that are adapted to site-specifically bind and photo-crosslink to the second antibody or antigen-binding fragment, wherein each of the second pair of pAbBDs is connected via a linker to the second binding pair member, and wherein the first and second binding pair members comprise two moieties that form a heterodimer; (c) contacting the first adapter comprising and the second adapter under conditions where the first and second binding pair members bind to each other; (d) site-specifically photo-crosslinking each of the first pair of pAbBDs to the first antibody or antigen-binding fragment to the first adapter; and (e) site-specifically photo-crosslinking the second antibody or antigen-binding fragment to the second adapter to form a bispecific antibody. In some embodiments, the method further comprises the step of purifying or isolating the bispecific antibody formed in step (e). In some embodiments, the method further comprises the step of purifying or isolating the product following step (c). In some embodiments, the method further comprises the step of purifying or isolating the product following step (d). In some embodiments, the two moieties that form a heterodimer form a covalently linked heterodimer. In some embodiments, the two moieties that form a heterodimer form a non-covalently linked heterodimer. In some embodiments, the two moieties that form a heterodimer are two protein moieties that form a heterodimer. In some embodiments, the two protein moieties that form a heterodimer form a covalently linked heterodimer. In some embodiments, the two protein moieties that form a heterodimer form a non-covalently linked heterodimer.

In another aspect, provided herein is a method of producing a bispecific antibody from a first antibody or antigen-binding fragment and a second antibody or antigen-binding fragment, the method comprising the steps of (a) providing an adapter comprising a first antibody binding domain (AbBD) fused to a second antibody binding domain (AbBD); (b) site-specifically linking the first antibody or antigen-binding fragment to the first AbBD; and (c) site-specifically linking the second antibody or antigen-binding fragment to the second AbBD to form a bispecific antibody. In some embodiments, steps (b) and (c) are performed simultaneously. In some embodiments, steps (b) and (c) are performed sequentially.

In another aspect, provided herein is a method of producing a bispecific antibody from a first antibody or antigen-binding fragment and a second antibody or antigen-binding fragment, the method comprising the steps of (a) site-specifically linking the first antibody or antigen-binding fragment to a first adapter comprising a first antibody binding domain (AbBD) attached to or modified with a first member of a binding pair to form a first adapter-antibody conjugate; (b) site-specifically attaching, modifying, or fusing a second antibody, antigen-binding fragment, or targeting ligand (e.g., aptamer) with a second member of the binding pair; and (c) contacting the first adapter-antibody conjugate and the second antibody, antigen-binding fragment, or targeting ligand under conditions where the first and second members of the binding pair bind to each other to form a bispecific antibody. In some embodiments, the first adapter and the second antibody, antigen-binding fragment, or targeting ligand are combined first, then the first antibody or antigen binding domain is site-specifically linked to the first adapter.

In another aspect, provided herein is a method of producing a bispecific antibody from a first antibody or antigen-binding fragment and a second antibody or antigen-binding fragment, the method comprising the steps of (a) site-specifically linking the first antibody or antigen-binding fragment to a first adapter comprising an antibody binding domain (AbBD) attached to or modified with a first member of a binding pair to form an adapter-antibody conjugate; (b) providing an antibody conjugate comprising the second antibody or antigen-binding fragment attached to or modified with a second member of the binding pair; and (c) contacting the adapter-antibody conjugate and antibody conjugate under conditions where the first and second members of the binding pair bind to each other to form a bispecific antibody.

In another aspect, provided herein are methods of producing a bispecific antibody from a first antibody or antigen-binding fragment and an antibody-adapter fusion comprising a second antibody or antigen-binding fragment fused to an adapter comprising an antibody binding domain (AbBD), the method comprising: site-specifically linking the first antibody or antigen-binding fragment to the antibody-adapter.

Also provided herein are bispecific antibodies produced according to the foregoing methods.

In another aspect, provided herein is a conjugate molecule or an adapter comprising a protein, such as a Protein G HTB1 domain or Protein Z domain, having one or more amino acids or amino acid modifications that are adapted to specifically bind and crosslink to an immunoglobulin. In another aspect, provided herein is a conjugate molecule or an adapter comprising a first antibody binding domain (AbBD) fused to a second antibody binding domains (AbBD), wherein the first AbBD has one or more amino acids or amino acid modifications that are adapted to specifically bind and crosslink to a first immunoglobulin and wherein the second AbBD has one or more amino acids or amino acid modifications that are adapted to specifically bind and crosslink to a second immunoglobulin.

Also provided herein are nucleic acids and vectors that encode the foregoing adapters. Further provided herein are cells that express the foregoing adapters.

In one embodiment, the immunoglobulin is IgG.

In another embodiment, the protein is a recombinant bacterial protein. In another embodiment, the recombinant bacterial protein is Protein Z.

In another embodiment, the recombinant bacterial protein is a subdomain of Protein G. In another embodiment, the recombinant bacterial protein is a subdomain of Protein A. In another embodiment, the recombinant bacterial protein is a Protein L or a subdomain thereof. In another embodiment, the recombinant bacterial protein is CD4 or a subdomain thereof.

In another embodiment the adapter is an antibody binding domain (AbBD).

In another embodiment, the antibody binding domain crosslinks to the immunoglobulin Fc region. In another embodiment, the antibody binding domain crosslinks to the immunoglobulin Fab region.

“Protein Z” refers to a Z domain based on the B domain of Staphylococcal aureus Protein A. The wild-type Protein Z amino acid sequence is: VDNKFNKEQQNAFYEILHLPNLNEEQRNAFIQSLKDDPSQSANLLAEAKKLNDAQAP KMRM (SEQ ID NO: 1). Photoreactive Protein Z includes those where an amino acid in protein Z has been replaced with benzoylphenylalanine (BPA), such as F13BPA and F5BPA (underlined amino acids in bold in SEQ ID NO: 1). Examples of other BPA-containing Protein Z mutants include, but are not limited to, Q32BPA, K35BPA, N28BPA, N23BPA, and L17BPA. Examples of Protein Z variants or mutants include, F5I, such as F5I K35BPA. The Protein Z amino acid sequence may also include homologous, variant, and fragment sequences having Z domain function. In some embodiments, the Protein Z amino acid sequence includes an amino acid sequence which is 60, 65, 70, 75, 80, 85, 90, 95, or 99% identity to the sequence set forth in SEQ ID NO: 1.

“Protein G” refers to a B1 domain based on Streptococcal Protein G. Preferably, the Protein G is a hyperthermophilic variant of a B1 domain based on Streptococcal Protein G. The Protein G amino acid sequence preferably is: MTFKLIINGKTLKGEITIEAVDAAEAEKIFKQYANDYGIDGEWTYDDATKTFTVTE (SEQ ID NO: 2). Nine Protein G variants were successfully designed and expressed, each with an Fc-facing amino acid substituted by BPA: V21, A24, K28, I29, K31, Q32, D40, E42, W42 (underlined and bold in SEQ ID NO: 23). Two variants, A24BPA and K28BPA, allowed ˜100% of all human IgG subtypes to be labeled. The Protein G amino acid sequence may also include homologous, variant, and fragment sequences having B1 domain function. In some embodiments, the Protein G amino acid sequence includes an amino acid sequence which is 60, 65, 70, 75, 80, 85, 90, 95, or 99% identity to the sequence set forth in SEQ ID NO: 2.

The “Fc domain” encompasses the constant region of an immunoglobulin molecule. The Fc region of an antibody interacts with various Fc receptors and ligands, imparting an array of important functional capabilities referred to as effector functions. For IgG, the Fc region comprises Ig domains CH2 and CH3. An important family of Fc receptors for the IgG isotype are Fc gamma receptors (FcγRs). These receptors mediate communication between antibodies and the cellular arm of the immune system.

A “Fab domain” encompasses an antibody region that binds to antigens. The Fab region is composed of one constant and one variable domain of each of the heavy and light chains.

The term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. The term “modified immunoglobulin G” refers to a molecule derived from an antibody of the “G” class. The term “antibody” refers to a protein of one or more polypeptides substantially encoded by all or part of a recognized immunoglobulin gene. The recognized immunoglobulin genes, for example in humans, include the kappa (κ), lambda (λ) and heavy chain genetic loci, which together comprise the myriad variable region genes, and the constant region genes mu (μ) delta (δ), gamma (γ), sigma (σ) and alpha (α) which encode the IgM, IgD, IgG, IgE, and IgA isotypes or classes, respectively. An antibody is meant to include full-length antibodies, and may refer to a natural antibody from any organism, an engineered antibody, or an antibody generated recombinantly for experimental, therapeutic, or other purposes. Furthermore, full-length antibodies encompass conjugates as described and exemplified herein. Antibodies may be monoclonal or polyclonal antibodies. Antibodies can be antagonists, agonists, neutralizing, inhibitory, or stimulatory. Specifically included as an “antibody” are full-length antibodies described and exemplified herein. By “full length antibody” herein is meant the structure that constitutes the natural biological form of an antibody, including variable and constant regions.

An antibody “variable region” contains the antigen binding determinants of the molecule, and thus determines the specificity of an antibody for its target antigen. The variable region is so named because it is the most distinct in sequence from other antibodies within the same isotype. The majority of sequence variability occurs in complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e., bi-specific) hybrid antibodies (e.g., Lanzavecchia et al., Eur. J. Immunol. 17:105 (1987)) and in single chains (e.g., Huston et al., Proc. Natl. Acad. Sci. 85:5879 (1988) and Bird et al., Science (1988) 242:423, which are incorporated herein by reference). (See, generally, Hood et al., “Immunology”, Benjamin, N.Y., 2nd ed. (1984), and Hunkapiller & Hood, Nature (1986) 323:15).

An “epitope” is a region of an antigen that binds to the antibody or antigen-binding fragment. It is the antigen region recognized by a first antibody where the binding of the first antibody to the region prevents binding of a second antibody or other bivalent molecule to the region. The region encompasses a particular core sequence or sequences selectively recognized by a class of antibodies. In general, epitopes are comprised of local surface structures that can be formed by contiguous or noncontiguous amino acid sequences.

The terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art, such as, for example, ELISA or cold displacement assays.

An antibody encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, Cγ1, Cγ2, and Cγ3. In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms including full-length antibodies, antibody fragments, and individual immunoglobulin domains including VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

The term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.
(6) scFv-Fc, is produced in one embodiment, by fusing single-chain Fv (scFv) with a hinge region from an immunoglobulin (Ig) such as an IgG, and Fc regions.

In some embodiments, the antibody is a monoclonal antibody. In some embodiments, the antigen-binding fragment is a single chain Fv (scFv), a diabody, a tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

The terms “bivalent molecule” or “BV” refer to a molecule capable of binding to two separate targets at the same time. A bivalent molecule is not limited to having two and only two binding domains and can be a polyvalent molecule or a molecule comprised of linked monovalent molecules. The binding domains of a bivalent molecule can selectively recognize the same epitope or different epitopes located on the same target or located on a target that originates from different species. The binding domains can be linked in any of a number of ways including disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983) (general review); Morley, J. S., Trends Pharm Sci (1980) pp. 463-468 (general review); Hudson et al., Int J Pept Prot Res (1979) 14:177; Spatola et al., Life Sci (1986) 38:1243; Hann, M., J Chem Soc Perkin Trans I (1982) 307-; Almquist et al., J Med Chem (1980) 23:1392; Jennings-White et al., Tetrahedron Lett (1982) 23:2533; Szelke et al., EP 45,665; Chemical Abstracts 97, 39405 (1982); Holladay et al., Tetrahedron Lett (1983) 24:4401; and Hruby, V. J., Life Sci (1982) 31:189).

The terms “binds” or “binding” or grammatical equivalents, refer to compositions having affinity for each other. “Specific binding” is where the binding is selective between two molecules. A particular example of specific binding is that which occurs between an antibody and antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

A “dimer” is a macromolecular complex formed by two macromolecules, usually proteins (or portions thereof) or nucleic acids (or portions thereof). A “homodimer” is formed by two identical macromolecules (“homodimerization”), while a “heterodimer” is formed by two distinct macromolecules (“heterodimerization”). Many dimers are non-covalently linked, but some (e.g., NEMO homodimers) can link via, e.g., disulfide bonds. Some proteins have regions specialized for dimerization, known as “dimerization domains.” In some cases, a truncated protein containing a dimerization domain (or two truncated proteins containing corresponding dimerization domains) may be able to interact in the absence of one or both complete protein sequence(s). Similarly, a fusion protein including a dimerization domain (or two fusion proteins including corresponding dimerization domains) may be able to interact in the absence of one or both complete protein sequence(s). Mutations to these domains may increase, or alternatively reduce, dimer formation.

In one embodiment, an antibody or antigen-binding fragment binds its target with a K_{D} of 0.1-10 mM. In one embodiment, an antibody or antigen-binding fragment binds its target with a K_{D} of 0.1-1 mM. In one embodiment, an antibody or antigen-binding fragment binds its target with a K_{D} within the 0.1 nM range. In one embodiment, an antibody or antigen-binding fragment binds its target with a K_{D} of 0.1-2 nM. In one embodiment, an antibody or antigen-binding fragment binds its target with a K_{D} of 0.1-1 nM. In one embodiment, an antibody or antigen-binding fragment binds its target with a K_{D} of 0.05-1 nM. In one embodiment, an antibody or antigen-binding fragment binds its target with a K_{D} of 0.1-0.5 nM. In one embodiment, an antibody or antigen-binding fragment binds its target with a K_{D} of 0.1-0.2 nM.

In some embodiments, the antibody or antigen-binding fragment thereof comprises a modification. In some embodiments, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody or other cellular ligand, etc. Any of numerous chemical modifications may be carried out by known techniques including specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In some embodiments, the modification is an N-terminal modification. In some embodiments, the modification is a C-terminal modification. In some embodiments, the modification is N-terminal biotinylation. In some embodiments, the modification is C-terminal biotinylation. In some embodiments, the secretable form of the antibody or antigen-binding fragment has an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment has an N-terminal modification that allows binding to an enzymatically biotinylatable site. In some embodiments, the secretable form of the antibody or antigen-binding fragment has a C-terminal modification that allows binding to an enzymatically biotinylatable site. In some embodiments, biotinylation of the site functionalizes the site to bind to a surface coated with streptavidin, avidin, avidin-derived moieties, or a secondary reagent.

It will be appreciated that a “modification” can encompass an amino acid modification such as an amino acid substitution, insertion, and/or deletion in a polypeptide sequence.

Various radioactive isotopes are available to produce radio-conjugate antibodies and other proteins that can be used in methods and compositions described here. Examples include At²¹¹, Cu⁶⁴, I¹³¹, I¹²⁵, Y⁹⁰, Re¹⁸⁶, Re¹⁸⁸, Sm¹⁵³, Bi²¹², P³², Zr⁸⁹ and radioactive isotopes of Lu.

In other embodiments, enzymatically active toxin or fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the protein, according to methods described herein, as an active drug or as a prodrug. A “prodrug” is a precursor or derivative form of a pharmaceutically active substance that is less cytotoxic to cells compared to the parent drug and is capable of being enzymatically activated or converted into the more active parent form. Prodrugs that may be used include phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug.

In one embodiment, a combination of the protein with the biological active agents specified above, i.e., a cytokine, an enzyme, a chemokine, a radioisotope, an enzymatically active toxin, or a chemotherapeutic agent can be applied.

A variety of other therapeutic agents may find use for administration with the antibodies and conjugates described herein. In one embodiment, the conjugate is administered with an anti-angiogenic agent. An “anti-angiogenic agent” refers to a compound that blocks, or interferes to some degree, with blood vessel development. It may, for instance, be a small molecule or a protein, for example an antibody, Fc fusion, or cytokine, that binds to a growth factor or growth factor receptor involved in promoting angiogenesis. In another embodiment, the conjugate is administered with a therapeutic agent that induces or enhances an adaptive immune response. In another embodiment, the conjugate is administered with a tyrosine kinase inhibitor. A “tyrosine kinase inhibitor” refers to a molecule that inhibits to some extent tyrosine kinase activity of a tyrosine kinase.

In one embodiment, conjugates described herein may be used for various therapeutic purposes. In one embodiment, the conjugates are administered to a subject to treat an antibody-related disorder. In another embodiment, the conjugates are administered to a subject to treat a tumor or a cancer tumor. A “subject” for the purposes described herein includes humans and other animals, preferably mammals and most preferably humans. The subject may include dogs, cats, pigs, cows, sheep, goats, horses, rats, and mice and humans. The term “subject” does not exclude an individual that is normal in all respects.

Thus, the conjugates provided herein have both human therapy and veterinary applications. In one embodiment the subject is a mammal, and in one embodiment the mammal is a human. A “condition” or “disease” includes a disorder that may be ameliorated by the administration of a pharmaceutical composition comprising a conjugate or by a method provided herein. Antibody related disorders include, but are not limited to, autoimmune diseases, immunological diseases, infectious diseases, inflammatory diseases, neurological diseases, and oncological and neoplastic diseases including cancer.

Provided herein are nucleic acid constructs encoding the adapters, conjugates and fusion proteins herein. A “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. The term includes oligonucleotides composed of naturally occurring nucleobases, sugars and covalent inter-nucleoside (backbone) linkages, as well as oligonucleotides having non-naturally-occurring portions, which function similarly. Such modified or substituted oligonucleotides are often preferred over native forms because of desirable properties, such as enhanced cellular uptake, enhanced affinity for nucleic acid target and increased stability in the presence of nucleases.

In some embodiments, the vectors comprise a nucleic acid encoding a protein, polypeptide, peptide, antibody, or recombinant protein described herein. In some embodiments, the vectors comprise nucleic acids encoding fusion proteins described herein.

In one embodiment, the nucleic acid can be expressed in a variety of different systems, in vitro and in vivo, according to the desired purpose. For example, a nucleic acid can be inserted into an expression vector, introduced into a desired host, and cultured under conditions effective to achieve expression of a polypeptide encoded by the nucleic acid. Effective conditions include culture conditions which are suitable for achieving production of the polypeptide by the host cell, including effective temperatures, pH, media, additives to the media in which the host cell is cultured (e.g., additives which amplify or induce expression such as butyrate, or methotrexate if the coding nucleic acid is adjacent to a dhfr gene), cycloheximide, cell densities, culture dishes, etc. In another embodiment, a nucleic acid is introduced into the cell by an effective method including, e.g., naked DNA, calcium phosphate precipitation, electroporation, injection, DEAE-Dextran mediated transfection, fusion with liposomes, association with agents which enhance its uptake into cells, viral transfection. A cell into which the nucleic acid has been introduced is a transformed host cell. The nucleic acid can be extrachromosomal or integrated into a chromosome(s) of the host cell. It can be stable or transient. An expression vector is selected for its compatibility with the host cell. Host cells include, mammalian cells (e.g., COS-7, CV1, BHK, CHO, HeLa, LTK, NIH 3T3, 293, PAE, human, human fibroblast, human primary tumor cells, testes cells), insect cells, such as Sf9 (S. frugipeda) and Drosophila, bacteria, such as E. coli, Streptococcus, bacillus, yeast, such as S. cerevisiae (e.g., cdc mutants, cdc25, cell cycle and division mutants, such as ATCC Nos. 42563, 46572, 46573, 44822, 44823, 46590, 46605, 42414, 44824, 42029, 44825, 44826, 42413, 200626, 28199, 200238, 74155, 44827, 74154, 74099, 201204, 48894, 42564, 201487, 48893, 28199, 38598, 201391, 201392), fungal cells, plant cells, embryonic stem cells (e.g., mammalian, such as mouse or human), fibroblasts, muscle cells, neuronal cells, etc. Expression control sequences are similarly selected for host compatibility and a desired purpose, e.g., high copy number, high amounts, induction, amplification, controlled expression. Other sequences that can be used include enhancers such as from SV40, CMV, RSV, inducible promoters, cell-type specific elements, or sequences which allow selective or specific cell expression. Promoters that can be used to drive expression, include an endogenous promoter, promoters of other genes in a cell signal transduction pathway, MMTV, SV40, trp, lac, tac, or T7 promoters for bacterial hosts; or alpha factor, alcohol oxidase, or PGH promoters for yeast.

In one embodiment, reporter genes are incorporated within expression constructs to facilitate identification of transcribed products. Accordingly, in one embodiment, reporter genes used are selected from the group consisting of β-galactosidase, chloramphenicol acetyl transferase, luciferase and a fluorescent protein.

In one embodiment, the conjugates are purified or isolated after expression. Proteins may be isolated or purified in a variety of ways known to those skilled in the art. Purification methods include chromatographic techniques, including ion exchange, hydrophobic interaction, affinity, sizing or gel filtration, and reversed-phase, carried out at atmospheric pressure or at high pressure using systems such as FPLC or HPLC. Purification methods also include electrophoretic, immunological, precipitation, dialysis, and chromatofocusing techniques. Ultrafiltration and diafiltration techniques, in conjunction with protein concentration, are also useful. As is known in the art, a variety of natural proteins bind Fc and antibodies, and these proteins can find use for purification of conjugates described herein. For example, the bacterial proteins A and G bind to the Fc region. Likewise, the bacterial protein L binds to the Fab region of some antibodies, as of course does the antibody's target antigen. Purification can often be enabled by a particular fusion partner. For example, proteins may be purified using glutathione resin if a GST fusion is employed, Ni⁺² affinity chromatography if a His-tag is employed, or immobilized anti-flag antibody if a flag-tag is used. The degree of purification needed will vary depending on the screen or use of the conjugates. In some instances, no purification is necessary. For example, if conjugates are secreted, screening may take place directly from the media. As known in the art, some selection methods do not involve purification of proteins. For example, if conjugates are made into a phage display library, protein purification may not be performed.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of 20% or 10%, more preferably ±5%, even more preferably ±1%, and still more preferably ±0.1% from the specified value, as such variations are appropriate to perform the disclosed methods.

Described herein are techniques for the rapid production of bispecific antibodies using full-length IgG. These techniques generally do not require any genetic manipulation of the IgG. Any off the shelf IgGs can be used to make the bispecific antibodies.

IgGs are site-specifically modified using photoreactive antibody binding domains. Antibody binding domains (AbBDs) include Protein A, Protein G, Protein L, CD4 and their subdomains, e.g., B1 domain of Protein G, or engineered subdomains, e.g., Protein Z, HTB1.

In some embodiments, one or more photo reactive groups, e.g., benzophenone, are introduced onto the AbBDs. These can be incorporated into the AbBDs during translation (e.g., benzoylphenylalanine, BPA) using non-natural amino acid incorporation or the AbBDs can be post-modified with a photocrosslinker (e.g., 4-(N-Maleimido)benzophenone). In this case a cysteine would be engineered into the AbBD at the location where the benzophenone is desired. BPA as a photoreactive crosslinker has several favorable properties. Specifically, BPA's benzophenone group can be activated by long wavelength UV light (365 nm), which is not harmful to antibodies or other proteins. Additionally, even after being UV excited to its triplet state, benzophenone can relax back to its unreactive ground state if there are no abstractable hydrogen atoms in close proximity. This allows the photoreactive proteins to be produced and handled in ambient light conditions with low risk of photobleaching. However, other photoreactive crosslinkers can also be used, including those that possess aryl azides, diazirines, or other photoreactive moieties known in the art.

To prepare bispecific antibodies, the AbBDs are fused or modified with a linking module or a member of a binding pair that allows two AbBDs to be linked together.

There are many options for linking modules. A variety of linkers may be used to generate conjugates and fusion proteins. The term “linker,” “linker sequence,” “spacer,” “tethering sequence” or grammatical equivalents thereof refer to a molecule or group of molecules (such as a monomer or polymer) that connects two molecules and often serves to place the two in a preferred configuration. Several strategies may be used to covalently link molecules together. These include, but are not limited to, polypeptide linkages between N- and C-termini of proteins or protein domains, linkage via disulfide bonds, and linkage via chemical cross-linking reagents. In one embodiment, the linker is a peptide bond, generated by recombinant techniques or peptide synthesis. In another embodiment, the linker is a cysteine linker. In yet another embodiment, it is a multi-cysteine linker. Choosing a suitable linker for a specific case where two polypeptide chains are to be connected depends on various parameters including, but not limited to, the nature of the two polypeptide chains (e.g., whether they naturally oligomerize) and the distance between the N- and C-termini to be connected, if known, and/or the stability of the linker towards proteolysis and oxidation. Furthermore, a linker may contain amino acid residues to provide flexibility. Thus, the linker peptide may predominantly include the amino acid residues: Gly, Ser, Ala, and Thr. The linker peptide should be adequately long to link two molecules in such a way that they assume the correct conformation relative to one another to retain the desired activity. Suitable lengths include at least one and not more than 30 amino acid residues. In one embodiment, the linker is from 1 to 30 amino acids long. In another embodiment, the linker is from 1 to 15 amino acids long. In addition, the amino acid residues selected should have properties that do not significantly interfere with the polypeptide's activity. Thus, the linker peptide overall should not have a charge inconsistent with the polypeptide's activity, or interfere with internal folding, or form bonds or other interactions with amino acid residues in one or more of the monomers that would seriously impede the binding of receptor monomer domains. Useful linkers include glycine-serine polymers, glycine-alanine polymers, alanine-serine polymers, and other flexible linkers such as the tether for the shaker potassium channel, and a large variety of other flexible linkers, as will be appreciated by those in the art. Suitable linkers may also be identified by screening databases of known three-dimensional structures for naturally occurring motifs that can bridge the gap between two polypeptide chains. In one embodiment, the linker is not immunogenic when administered in a human. Thus, linkers may be chosen such that they have or are thought to have low immunogenicity. Another way of obtaining a suitable linker is to optimize a simple linker, e.g., (Gly₄Ser)ₙ, (SEQ ID NO: 20), through random mutagenesis. Alternatively, once a suitable polypeptide linker is defined, additional linker polypeptides can be created to select amino acids that more optimally interact with the domains being linked. Other types of linkers that may be used include artificial polypeptide linkers and inteins. In another embodiment, disulfide bonds are designed to link two molecules. In another embodiment, linkers are chemical cross-linking agents. For example, a variety of bifunctional protein coupling agents may be used, such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In another embodiment, chemical linkers may allow chelation of an isotope. Carbon-14-labeled 1-isothiocyanatobenzyl-3-methyldiethylene triaminepentaacetic acid (MX-DTPA) is an exemplary chelating agent to conjugate a radionucleotide to an antibody. The linker may be cleavable, facilitating release of the cytotoxic drug in the cell. For example, acid-labile, peptidase-sensitive, dimethyl linker or disulfide-containing linkers (Chari et al., Cancer Research (1992) 52:127) may be used. Alternatively, various nonproteinaceous polymers, such polyethylene glycol (PEG), polypropylene glycol, polyoxyalkylenes, or copolymers of polyethylene glycol and polypropylene glycol, may find use as linkers. In one aspect, the invention provides biological linking modules. These are fused in frame with the AbBDs at the N- or C-terminus.

### Binding Pairs:

SpyCatcher and SpyTag. For example, One AbBD can be fused to SpyCatcher and a second AbBD can be fused to SpyTag. See Zakeri et al., “Peptide tag forming a rapid covalent bond to a protein, through engineering a bacterial adhesin” PNAS (2012) vol. 109 no. 12, pgs. E690-E697, doi: 10.1073/pnas.1115485109, which is hereby incorporated by reference in its entirety.

The first generation SpyCatcher amino acid sequence is: ATHIKFSKRDEDGKELAGATMELRDSSGKTISTWISDGQVKDFYLYPGKYTFVETAA PDGYEVATAITFTVNEQGQVTVN (SEQ ID NO: 17). The SpyCatcher amino acid sequence may also include homologous, variant, and fragment sequences having SpyCatcher function. In some embodiments, the SpyCatcher amino acid sequence includes an amino acid sequence which is 60, 65, 70, 75, 80, 85, 90, 95, or 99% identity to the sequence set forth in SEQ ID NO: 17.

The first generation SpyTag amino acid sequence is: AHIVMVDAYKPTK (SEQ ID NO: 18). The SpyTag amino acid sequence may also include homologous, variant, and fragment sequences having SpyTag function. In some embodiments, the SpyTag amino acid sequence includes an amino acid sequence which is 60, 65, 70, 75, 80, 85, 90, 95, or 99% identity to the sequence set forth in SEQ ID NO: 18. Variants of SpyCatcher and SpyTag are known, for example, Variants of SpyCatcher and SpyTag are described, including their amino acid sequences, in Keeble et al., PNAS (December 2019) 116 (52) 26523-26533 and See Hatlem et al., Int. J. Mol. Sci. 2019, 20, 2129, each of which is hereby incorporated by reference in its entirety.

SnoopCatcher and SnoopTag. For example, one AbBD can be fused to SnoopCatcher and a second AbBD can be fused to SnoopTag. See Hatlem et al., Int. J. Mol. Sci. 2019, 20, 2129, which is hereby incorporated by reference in its entirety.

Split inteins (or other intein-based systems). One AbBD can be fused to one half of the split intein and the other AbBD can be fused to the other half.

Heterodimeric proteins that have an affinity for each other (e.g., c-fos and c-jun, leucine zippers, peptide velcro, etc.) can also be used.

Dock-and-lock. This system involves two docking proteins, which are fused to the AbBDs. These proteins bring together the two AbBDs. Then a third peptide is used to covalently link the two docking proteins together.

Sortase. Sortase substrates (e.g., LPXTG (SEQ ID NO: 21) and an N-terminal glycine) are fused to the AbBDs and then free or fused sortase is used to ligate the two AbBDs together.

In another aspect, the invention provides chemical linking modules. The AbBDs are modified at their N- or C-terminus with various chemical moieties that can be used to link them together.

Click chemistries. One AbBD can be modified with an azide and the other with an alkyne or constrained alkyne (e.g., ADIBO or DBCO). Other popular click chemistries exist (e.g., tetrazine and TCO). Click chemistries can be incorporated using various techniques, e.g., intein-mediated expressed protein ligation, sortase, sortase-tag expressed protein ligation, non-natural amino acid incorporation, maleimide chemistry, carbodiimide chemistry, NHS chemistry, aldehyde chemistry, chemoenzymatic approaches (e.g., lipoic acid ligase, formylglycine), etc. Similarly, one binding pair member can be or can contain an azide and the other binding pair member can be or have an alkyne or constrained alkyne (e.g., ADIBO or DBCO) and the heterodimer can be formed by click chemistry.

In one aspect, the invention provides oligonucleotides. Click chemistries or conventional chemistries are used to attach oligonucleotides (e.g., complementary oligonucleotides) to the AbBDs. The oligonucleotides are then used to bring together (e.g., by hybridization) the two AbBDs. Additionally, one binding pair member can be an oligonucleotide and the other binding pair member can be a complementary oligonucleotide and the heterodimer can be formed by hybridization of the two oligonucleotides.

AbBDs with complementary linking modules (e.g., SpyCatcher and SpyTag) are covalently linked to IgG upon exposure to long UV light (typically long wavelength UV light). The two complementary AbBD-IgG conjugates are then mixed together to form the bispecific antibody.

In other embodiments, a single construct with two photoreactive AbBDs fused together are used to make bispecific antibodies. For example, photoreactive AbBDs with unique specificity for different IgG isotypes are fused. Therefore, if it is desirable to link together two IgGs with two distinct subclasses, it is not necessary to use a linking module; rather AbBDs that are directly fused together can be used.

Similarly, in other embodiments, IgG homodimers are prepared using AbBDs that are fused together and do not require a linking module.

While certain methods herein are exemplified by making bispecific antibodies, the methods provided here are not limited to making antibody-antibody conjugates. It will be appreciated that the methods provided herein can also be used to make antibody-protein and antibody-enzyme conjugates, as well as other types of antibody-conjugates. In these cases, the second linking module is placed on the protein or enzyme that is to be linked to the AbBD-IgG conjugate, which contains the other half of the linking module, e.g., to make IgG-affibody conjugates.

AbBDs typically bind both heavy chains on IgG. The present methods include techniques to limit AbBD binding to one heavy chain per IgG. Alternatively, IgG with only a single AbBD attached can be isolated. The present methods also include techniques where an adapter has a pair of AbBDs that bind to both heavy chains in a single IgG molecule.

Pharmaceutical compositions are contemplated wherein fusion conjugate or adopter of the compositions and methods provided herein and one or more therapeutically active agents are formulated. Formulations of the conjugates of the compositions and methods provided herein are prepared for storage by mixing said antibody having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g., Zn-protein complexes); and/or non-ionic surfactants or polyethylene glycol (PEG). In another embodiment, the pharmaceutical composition that comprises the conjugate of the compositions and methods provided herein is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

The conjugate molecules disclosed herein may also be formulated as immunoliposomes. A liposome is a small vesicle comprising various types of lipids, phospholipids and/or surfactant that is useful for delivery of a therapeutic agent to a mammal. Liposomes containing the conjugates are prepared by methods known in the art, such as described in Epstein et al., 1985, PNAS, 82:3688; Hwang et al., 1980, PNAS, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The conjugate molecules provided herein may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers (which are injectable microspheres composed of lactic acid-glycolic acid copolymer and leuprolide acetate), and poly-D-(−)-3-hydroxybutyric acid) which is a microsphere-based delivery system composed of the desired bioactive molecule incorporated into a matrix of poly-DL-lactide-co-glycolide (PLG).

The conjugate molecules may also be linked to the surfaces of nanoparticles using the linking methods provided herein. In one embodiment, the nanoparticles can be used for imaging or therapeutic purposes.

Administration of the pharmaceutical composition comprising the conjugates provided herein, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. As is known in the art, the pharmaceutical composition may be formulated accordingly depending upon the manner of introduction.

Any patent, patent application publication, or scientific publication, cited herein, is incorporated by reference herein in its entirety.

The following examples are presented to illustrate preferred embodiments more fully. They should in no way be construed, however, to limit the invention's broad scope.

### EXAMPLES

### Example 1: Facile Method for the Site-Specific, Covalent Attachment of Full-Length IgG

Bispecific antibodies: Bispecific antibodies have emerged as a highly promising treatment for cancer. Bispecific antibodies physically bring T-cells and cancer cells closer together to enhance cancer cell killing. Perhaps best demonstrating the promise of bispecific antibody is blinantumomab, an anti-CD3×anti-CD19 pair, which has produced clinical remission in precursor B cell acute lymphoblastic leukemia (B-ALL) at thousand fold lower dosages than rituximab (anti-CD20 monoclonal antibody) and doing so without needing a secondary T-cell stimulatory signal. Similarly, Catumaxomab, has led to clinical benefit against malignant ascites with just four intraperitoneal infusions totaling 230 gg over 11 days. Conventional antibody therapies require cumulative antibody amounts ranging from 5-20 g per patient and years of therapy. Given these successes, bispecific antibodies can be a paradigm-shifting therapeutic for cancer treatment.

Challenges associated with bispecific antibodies: Despite the promise of bispecific antibodies, there are many challenges associated with their use, starting with their production. Early methods involved chemically crosslinking two IgGs, which unsurprisingly produced aggregates and very low amounts of functional product. Later, quandroma technology that fused two hydridomas was used. However, due to the random association between the various light and heavy chains, only ˜⅛ of the resulting antibodies had the desired dual specificity. Newer technologies have enabled two scFv's to be fused and recombinantly produced. One of the most promising classes of tandem scFv's is termed bispecific T cell engager (BiTE), which includes blinantumomab. For agents generated by recombinant engineering (e.g., BiTEs, diabodies, tandem scFvs, dock and lock), limitations include significant amounts of designing and cloning up front to even generate a product, low yields, instability in serum, formation of aggregates or dissociated subunits, reduced functional activity or binding affinity/avidity attributed to steric factors or altered conformations, etc. Agents that lack a constant region also typically exhibit a short in vivo half-life (thus requiring continuous infusion), as well as complete loss of Fe-related effector functions (e.g., ADCC, CDC, and binding to neonatal Fc receptors).

One of the most clinically successful classes of bispecific antibody is Triomab (e.g., Catumaxomab), which is composed of mouse IgG2a and rat IgG2b. Mouse IgG2a and rat IgG2b demonstrate a species-restricted heavy/light chain pairing and result in the production of correct full-length IgG. Despite the high immunogenicity of this rat/mouse hybrid molecule, it does not constitute a major issue possibly due to the small amounts administered (˜100 μg, compared to 3 g for rituximab), the short duration of the treatment (ten days) and the IP route of administration. However, intravenous (IV) injections will be required for other indications. In a phase 1 study for the treatment of non-small cell lung cancer, it was established that the maximum tolerated dose for multiple Triomab IV administration was only 5 g, together with a pre-medication of dexamethasone and antihistamines. This may limit the broad applicability of Triomab in treating solid tumors.

Adding to the challenges in producing bispecific antibodies is an incomplete understanding of their mode of action. For example, not all anti-CD3 antibodies work equally well to trigger T-cell activation. Given the high cost and time required for production, a methodology that allows bispecific antibodies to be produced from unmodified full-length IgG rapidly and cost-effectively would be highly valuable. We have developed facile methods for the efficient production of bispecific antibodies that will fulfill this need. Since bispecific antibodies may be produced from full-length unmodified IgG, no antibody engineering or cloning is required. Further, bispecific antibodies can be produced with high purity in as little as one day.

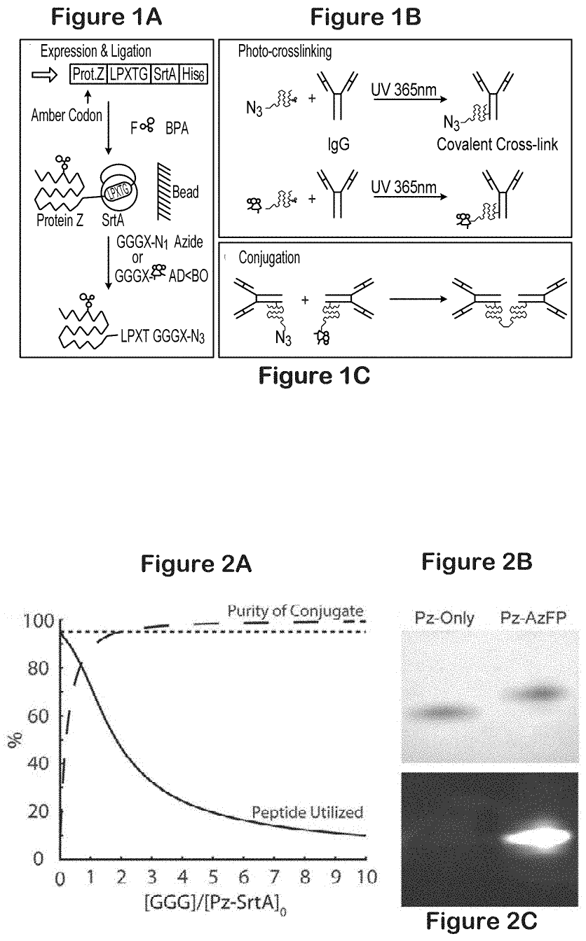

Bispecific antibodies can be produced by leveraging two complementary technologies, unnatural amino acid mutagenesis and expressed protein ligation, e.g., sortase-tag expressed protein ligation (STEPL). These techniques are described in greater detail in U.S. Appl. No. 61/799,379 (filed Mar. 15, 2013), in PCT Appl. No. PCT/US2014/030208 entitled “Sortase-Mediated Protein Purification and Ligation” (filed Mar. 17, 2014), in U.S. Appl. No. 61/800,926 (filed Mar. 15, 2013), and in PCT Appl. No. PCT/US2014/030457 entitled “Method for the Site-Specific Covalent Cross-Linking of Antibodies to Surfaces” (filed Mar. 17, 2014), each of which is hereby incorporated by reference in its entirety. These technologies are combined to recombinantly produce an antibody-binding domain derived from protein Z or protein G with two key features: (1) a photo-crosslinker (benzoylphenylalanine, BPA) within the antibody binding domain and (2) an azide or constrained alkyne moiety (e.g., azadibenzocyclooctyne, ADIBO) at the c-terminus (FIG. 1A). The photocrosslinker allows for covalent linkage to the Fc domain of IgG (FIG. 1B). This prevents protein Z/G from dissociating from the antibody following administration. The azide and constrained alkyne moieties allow the antibody-protein Z/G complexes to be site-specifically and efficiently linked via click chemistry (FIG. 1C). Notably, the formation of homodimers is not possible with this strategy, and the presence of only a single azide or ADIBO moiety on each antibody prevents the formation of aggregates/oligomers.

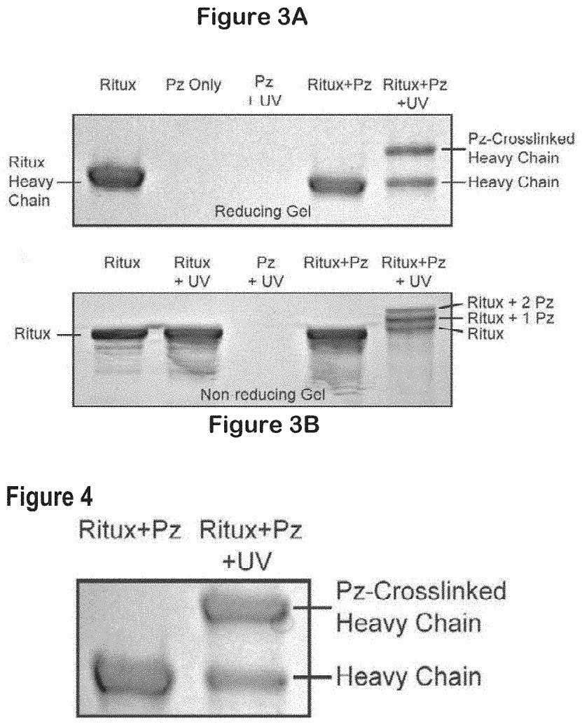

Sortase-Tag Expressed Protein Ligation (STEPL): Sortase A (SrtA) is a calcium-assisted transpeptidase that is responsible for anchoring surface proteins to the peptidoglycan cell wall of Gram-positive bacteria. The enzyme cleaves the peptide bond between the amino acids T and G, within the motif, LPXTG (SEQ ID NO: 21). The products remain transiently attached to SrtA, until the N-terminal glycine of another protein displaces the C-terminal fragment and forms a new peptide bond between the two-peptide chains. Recently, we created a single fusion protein construct with LPXTG (SEQ ID NO: 21), SrtA, and a His-tag, respectively, fused to the C-terminal end of Protein Z (FIG. 1A). The Protein Z is released from the affinity column upon ligation to a synthetic peptide with an N-terminal glycine. To produce bispecific antibodies, the peptide is also be labeled with a c-terminal azide or ADIBO. STEPL is site-specific and stoichiometric (i.e 1 azide or ADIBO per protein Z). The purity of the desired conjugate (i.e. Protein Z-azide/ADIBO) is >95% using only a two-fold excess of glycine-azide/ADIBO peptide, per Protein Z (FIG. 2). Therefore, very little excess peptide is required, keeping production cost low. Importantly, this STEPL system links protein purification and conjugation into a single step. Therefore, no post-modification steps are required to label Protein Z with an azide or ADIBO, outside of standard protein purification protocols.

In vivo incorporation of benzoylphenylalanine (BPA) during protein expression: The coding sequence for wild-type Protein Z sequence was cloned into the STEPL-compatible plasmid. To allow for incorporation of the unnatural amino acid, BPA, during translation, site-directed mutagenesis was performed to introduce an amber codon into the IgG binding site of Protein Z (FIG. 1A). Host E. coli were co-transformed with the plasmids encoding for photoreactive protein Z or wild-type protein Z and the pEVOL-pBpF plasmid (Addgene), which carries the tRNA/aminoacyl transferase pair. While wild-type Protein Z-STEPL fusion could be expressed in the absence of BPA, the mutant containing the amber codon required BPA for expression. There was no “leaky” background incorporation of other amino acids in response to the amber codon and the expression level for the BPA-containing mutant protein was comparable to that of the wild type Protein Z.

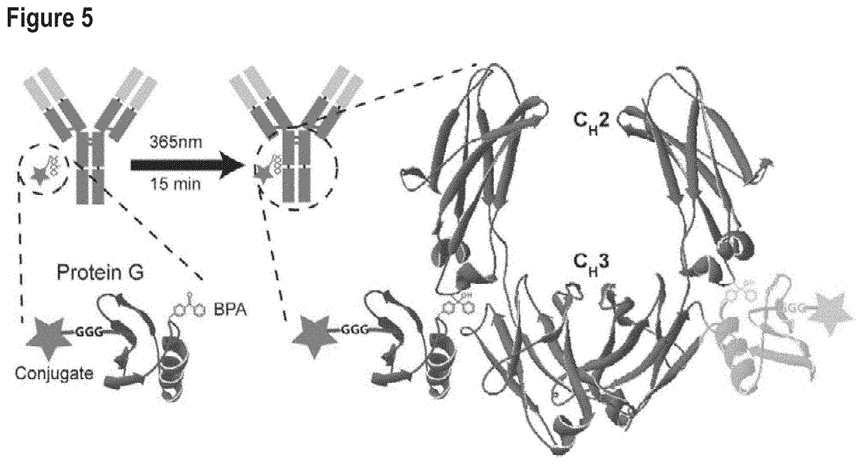

Protein Z-antibody crosslinking: To evaluate the crosslinking capabilities of photoreactive Protein Z, the BPA-protein Z variant was incubated with the humanized IgG1 monoclonal antibody rituximab and exposed to long wavelength UV light (365 nm) for 30 min (FIG. 1B). The extent of crosslinking was assessed via reducing and non-reducing SDS-PAGE gels (FIG. 3). In the reducing gel, one additional band was observed above the heavy chain band, corresponding to Protein Z-crosslinked heavy chain. More than 50% of the heavy chains appear to be crosslinked. On the non-reducing gel, two additional bands are observed in the crosslinked rituximab sample, compared with the non-crosslinked sample. These bands correspond to IgG crosslinked with one or two Protein Zs (FIG. 3B). Image analysis of the non-reducing gels show that 60-80% of rituximab is crosslinked with at least one Protein Z. The binding of a single Protein Z construct eliminates the formation of antibody oligomers, when the azide and ADIBO labeled antibodies are mixed.

Benefits of this approach: One of the advantages of this bispecific antibody production method is that any “off-the-shelf,” Protein Z-compatible full-length antibody can be used with no need for protein engineering, cloning, or other modifications. Moreover, Protein Z-IgG crosslinking is extremely rapid (˜30 min) and efficient. Therefore, this technique is amenable to high-throughput production, which is not currently possible with other techniques. This may allow for rapid screening of bispecific antibody pairs (e.g., different targets, different epitopes, different affinities) for optimal performance. Other advantages include the ability to swap between murine and human antibodies that target the same epitope, genetically modify Protein Z/G to alter immunogenicity, add additional functionality—toxins, imaging agents, drugs, radiopharmaceuticals or other chemical modifications can easily be added to the peptide used for STEPL (or other expressed protein ligation technique)—and there is an opportunity to expand the approach to trimeric, tetrameric, and higher order antibody conjugates by implementing multiple orthogonal click chemistries. Alternatively, IgG can be attached to alternative targeting ligands (e.g., scFv's, affibodies, etc.) or enzymes.

### Example 2: Light Activated Site-Specific Conjugation (LASIC) of Native IgGs

Numerous biological applications, from diagnostic assays to immunotherapies, rely on the use of antibody-conjugates. The efficacy of these conjugates can be significantly influenced by the site at which Immunoglobulin G (IgG) is modified. Current methods that provide control over the conjugation site, however, suffer from a number of shortfalls and often require large investments of time and cost. We have developed a novel adapter protein that, when activated by long wavelength UV light, can covalently and site-specifically label the Fc region of nearly any native, full-length IgG, including all human IgG subclasses. Labeling occurs with unprecedented efficiency and speed (>90% after 30 min), with no effect on IgG affinity. The adapter domain can be bacterially expressed and customized to contain a variety of moieties (e.g., biotin, azide fluorophores), making reliable and efficient conjugation of antibodies widely accessible.

Monoclonal antibodies, because of their broad repertoire of targets and exquisite selectivity, have become an essential component for a wide range of biological applications, from diagnostic assays to immunotherapies. Many of these applications require Immunoglobulin G (IgG) to be modified with a chemical (e.g., biotin, contrast agent, drug, nanoparticle) or biological agent (e.g., enzyme, second antibody).

While these diverse antibody formats are commonplace, their complex structures still pose various developmental and production challenges. A salient hurdle involves how to attach the functional moiety at specific locations away from the binding pocket of the antigen binding Fab domain, so as to preserve binding affinity and obtain homogenous products. Site-specific modifications have been widely shown to improve the performance and efficacy of antibody-conjugates in almost every known application.

Several enzymatic and recombinant based approaches have been utilized to enable the site-specific modification of IgG; however, these methods are lengthy and expensive, and often require cloning and cell line development for each construct. Despite the exploding interest in site-specifically modified antibody conjugates, these barriers limit their production to specially equipped labs and severely constrain the number and types of conjugates that can be made. This not only prevents the use of optimal antibody constructs for common laboratory assays, but also stunts the discovery and exploration of new antibody-based therapeutics, and hampers our understanding into the mechanisms of actions of these new formats.

A better approach for developing antibody conjugates would take advantage of the large library of existing antibodies. A means to conjugate existing native antibodies site-specially, rapidly and inexpensively can become an enabling technology to further antibody conjugate discovery and design. We have developed such as a platform, termed LASIC (Light Activated SIte-specific Conjugation) that enables highly efficient and versatile conjugation of nearly all IgGs, including all human subtypes.

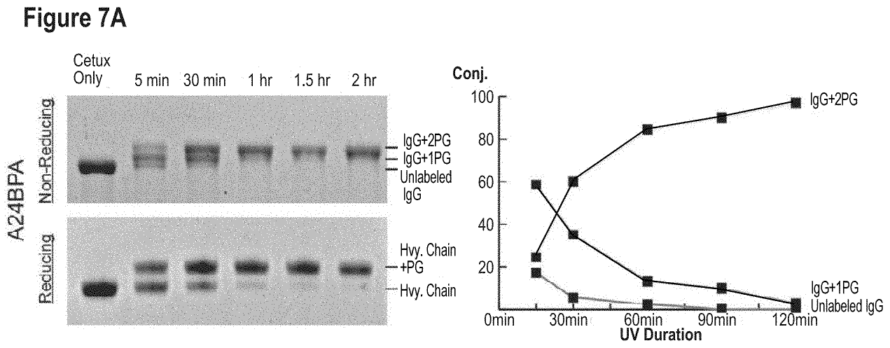

LASIC uses a small adapter protein that is engineered to contain the photoreactive non-natural amino acid benzoyl-phenylalanine (BPA) in its IgG binding domain, as well as a customizable reactive moiety at its C-terminus (FIG. 5). While we previously developed an adapter protein based on Protein A, it showed moderate to no conjugation towards human IgG subtypes. We therefore reasoned that the more broadly binding Protein G might serve as a better platform for LASIC. Protein G is derived from Streptococcal bacteria and can naturally bind to a broad range of IgGs at the CH2-CH3 junction. However, the non-covalent nature of the association between Protein G and IgG makes it ill-suited for making antibody conjugates. Although covalently linking Protein G onto IgG has been done using both chemical and photo-activated means, these methods were plagued either by decreased IgG affinity or by complex production and poor efficiency.

LASIC adapters, which possess a BPA crosslinker only in the Fc-binding domain, gives homogeneous products by forming only one covalent bond with IgG, rather than randomly labeling lysines as is the case with chemical crosslinking (FIG. 5). In addition, by recombinantly producing LASIC adapters using a well-established E coli. expression system that can incorporate BPA into proteins via an amber-tRNA suppressor aminoacyl-synthase pair, adapters with BPA in different locations can be efficiently produced and tested against different antibodies.

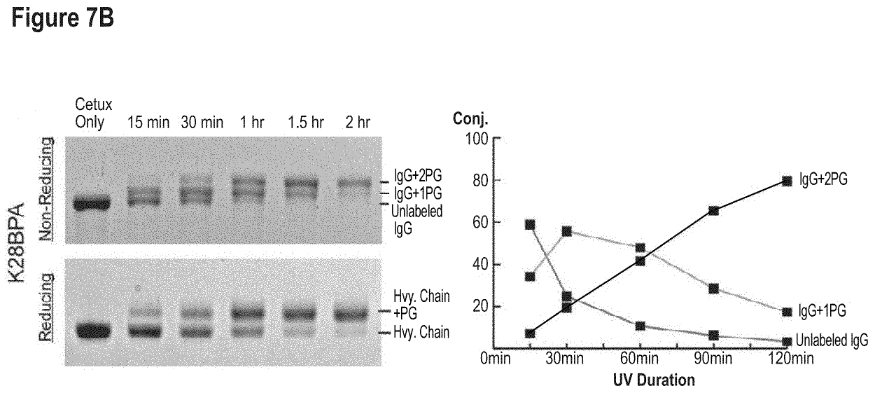

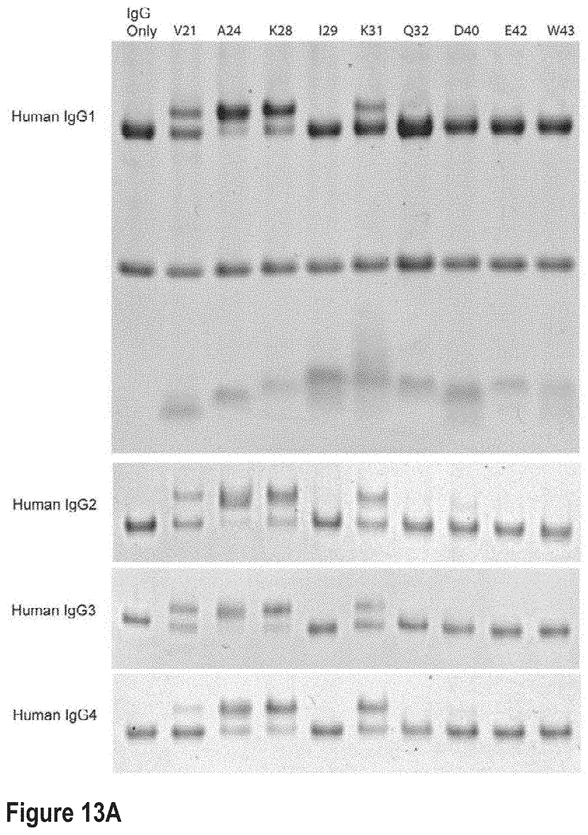

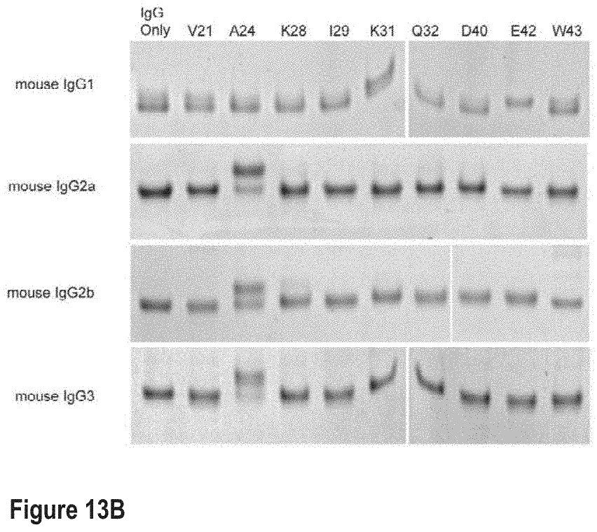

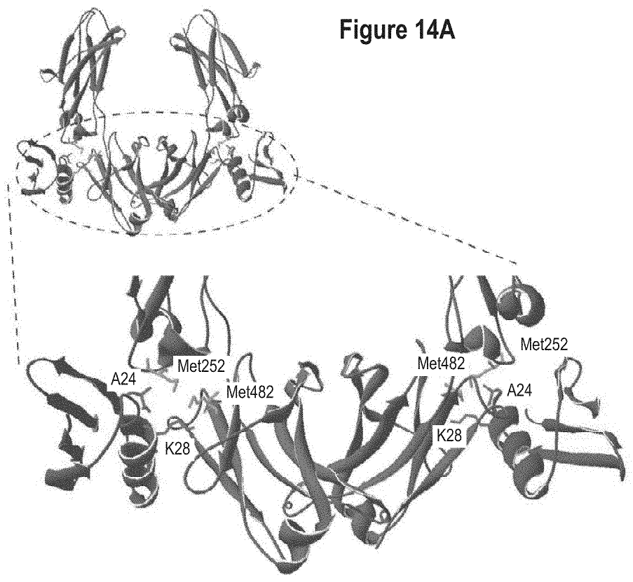

To minimize the “footprint” of the LASIC adapter and to ensure Fc-specific conjugation, we chose to use a small (6.5 kD), thermally stable domain of Protein G (HTB1), with a mutation to disable Fab-binding, as the parental molecule. We successfully designed and expressed nine Protein G variants, each having an Fc-facing amino acid substituted by BPA: V21, A24, K28, I29, K31, Q32, D40, E42, W42 (FIG. 12). The yields of expression for all variants were high at around 5 mg/mL, consistent with previous reports of BPA incorporation into proteins. Next, we screened these variants for their ability to covalently label a range of IgG isotypes from various hosts, upon exposure to long wavelength UV light (FIG. 13A-C). Since each IgG is composed of two identical heavy chains, it can be labeled with up to two Protein G-based adapters, which can be deciphered using non-reducing SDS-PAGE. We found two variants, A24BPA and K28BPA, that allowed ˜100% of all human IgG subclasses to be labeled with at least one adapter protein (FIG. 6). More than 90% of all human IgG subtypes were labeled with two adapter proteins (i.e. one adapter protein per heavy chain). In addition, A24BPA is also capable of conjugating most mice (mIgG 2a,2b,2c,3) as well as some rat and rabbit subtypes (rat 2c, rabbit polyclonal) with similar efficiencies (FIG. 14B, C). It has been known that BPA preferentially crosslinks methionine residues. Indeed, a three-dimensional model of the IgG-Protein G complex shows that A24 and K28 come in very close proximity to Met252 and Met482 on IgG, respectively (FIG. 14A). In fact, Met252 is found on all IgG that are efficiently labeled with A24BPA, while the same applies for Met428 and K28BPA (FIG. 14B).

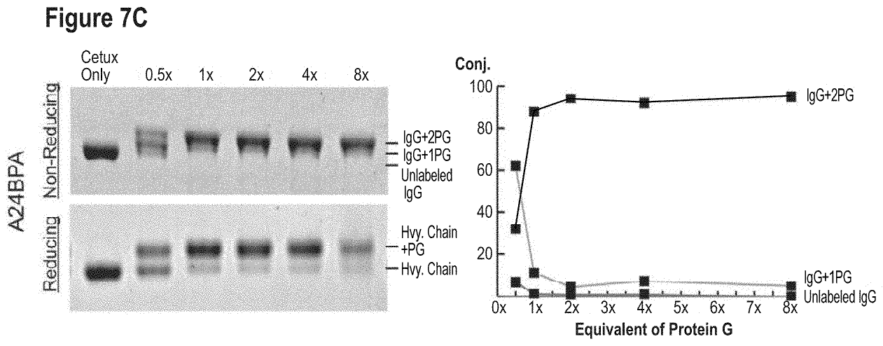

LASIC using A24BPA and K28BPA demonstrated unprecedented fast kinetics. After only 15 minutes of light exposure, more than 80% of IgG were conjugated by one or two A24BPA adapters, while the level reached 95% by 30 minutes (FIG. 7A). K28BPA reacted quickly as well, reaching 75% and 90% conjugation after 30 minutes and 1 hour respectively. The reaction was nearly stoichiometric with complete conjugation of IgG using only one equivalent of A24BPA (FIG. 7B). The fast conjugation kinetics by LASIC adapters is a significant improvement over the performance reported previously using with photoactive protein A or Protein G, where only around 50% of human IgG1 and IgG4 were conjugated after one hour, and negligible or no conjugation was seen for human IgG2 and IgG3, respectively. Similar conjugation efficiencies are reproducible for different IgGs of the same isotype. Similar results are also achievable using other readily available UV light sources and in a variety of common buffers (data not shown).

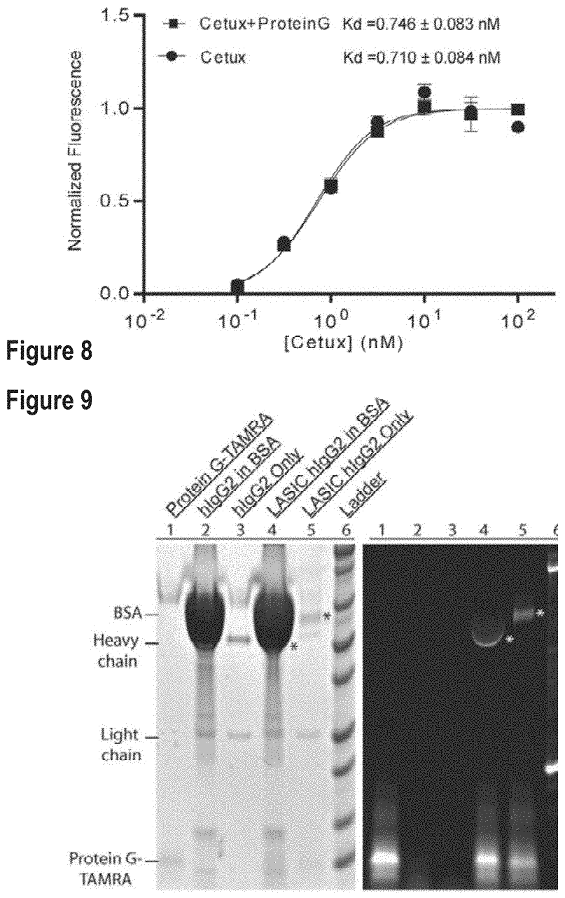

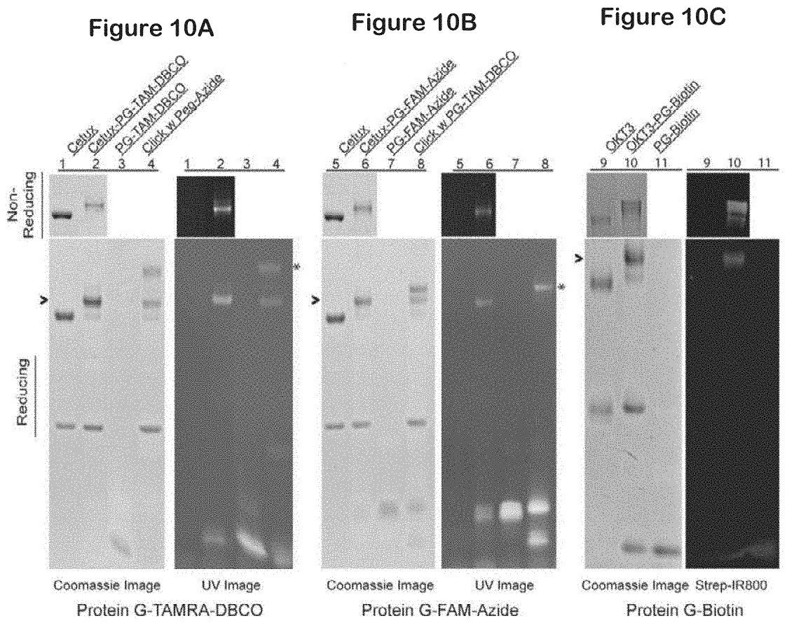

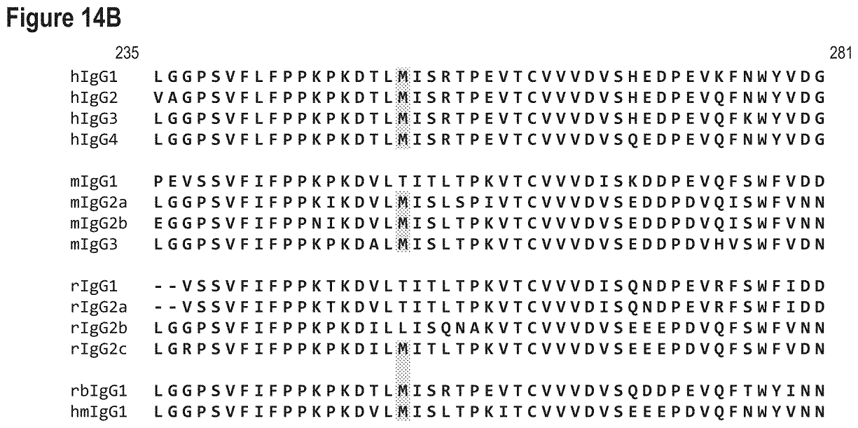

The structural stability of the Protein G HTB1 domain gives LASIC adapters a long shelf life even at room temperature, with no detectable loss of activity even after weeks of storage (FIG. 15). The use of BPA, which is only activated by non-harmful long wavelength UV light (365 nm) and is only quenched if in close proximity to a target (10 Å) with which it can form a covalent bond, makes the LASIC adapter safe to use, stable under ambient light, and non-reactive towards other proteins that it cannot bind (FIG. 15). To demonstrate the preservation of antigen binding after LASIC, we first conjugated the human IgG1 anti-human EGFR antibody (cetuximab) with the A24BPA adapter. Next we applied either unmodified cetuximab or LASIC treated cetuximab to EGFR-positive KB cells followed by detection using a fluorescent anti-human secondary antibody. Analysis of the fluorescent signals by a plate-reader indicated that both the unmodified and LASIC treated cetuximab showed similar binding affinity to the target cell line, demonstrating the gentle nature of photo-conjugation (FIG. 8). LASIC's exquisite specificity towards IgG allows conjugation to be done even in the presence of other proteins. This was shown by labeling hIgG2, either by itself or in 1% BSA solution, with a TAMRA (5-Carboxytetramethylrhodamine) dye-tagged LASIC adapter, followed by analysis using reducing SDS-PAGE gel (FIG. 9). While similarly high level of IgG2 heavy chains were labeled by the Protein G with or without BSA, as determined from the fluorescent image, none of the BSA was labeled despite being present at more than 200 times molar excess.

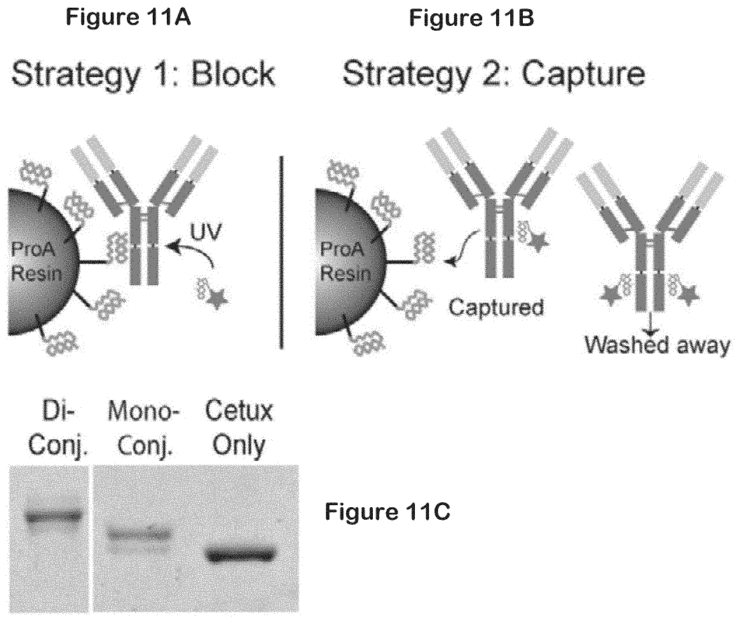

In order to produce LASIC adapters with a variety of C-terminal modifications we used the sortase expressed protein ligation (STEPL) technology, developed in our lab, to incorporate various moieties during the recombinant protein purification process. To demonstrate the versatility of this approach, we introduced three different Gly-Gly-Gly N-terminated peptides containing either a biotin, a 5-TAMRA dye along with a dibenzocyclooctyl (DBCO), or a 5-FAM (5-Carboxyfluorescein) dye along with an azide. The resulting adapters were then photo-conjugated to IgG (FIG. 10). As assayed by SDS-PAGE, nearly all of the heavy chains of IgGs were conferred with the functionalities carried by their respective Protein G adapters. There was no decrease in the conjugation efficiency as the moieties are on the C-terminus of the LASIC adapter and hence do not interfere with IgG binding. Since N-terminal triglycine peptides can be quickly and inexpensively synthesized, other reactive groups can be efficiently conjugated onto IgGs just as easily using LASIC.

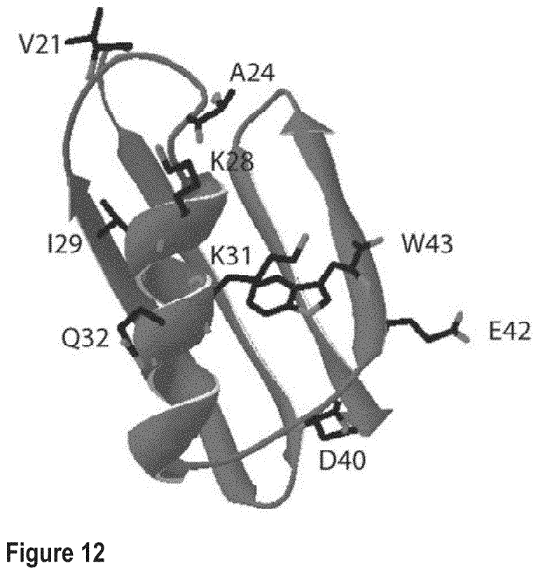

One feature of using a Protein G-based adapter is that both IgG heavy chains can be modified. While this may be preferred when maximum conjugation is important, in some instances it may be desirable to introduce only a single modification onto IgG. With LASIC it is possible to obtain mono-conjugated IgGs by slightly altering the conjugation or purification protocol (FIG. 11). Since the Fc site bound by LASIC adapters overlaps with the natural binding site of wild-type Protein G and Protein A, pre-adsorbing the IgG onto either Protein A or Protein G resin effectively blocks one of the two heavy chains, therefore giving only one conjugate per IgG after LASIC treatment. A similar approach involves absorbing IgG onto resins containing the photoreactive antibody binding domains. Then after photocrosslinking the adapter-IgG conjugates can be released from the resin using various methods known in the art, including the STEPL approach. Alternatively, as di-conjugated products cannot bind Protein A or Protein G, mono-conjugated product can also be captured from a mixture of di- and mono-conjugated IgGs using Protein A or Protein G resin. The ability to control the number of conjugates on the IgG affords an additional level of control, by allowing, for example, one to tailor the drug to antibody ratio when making antibody-drug conjugates (ADCs). Additionally, the ability of mono-conjugated product to bind to Protein A and G columns also greatly eases the purification of these conjugates. Furthermore, mono-conjugated IgG leaves one Fc-receptor binding site available for natural effector functions, including antibody dependent cell-mediated cytotoxicity (ADCC) and FcRn-mediated IgG recycling.

In summary, we have demonstrated that by using a recombinant Protein G-based adapter, one can efficiently photo-conjugate IgGs with a variety of moieties. Given the tremendous potential of site-specific antibody conjugates, there is a need for generating them more efficiently, ideally from full length IgGs so as to take advantage of their existing vast library, validated binding properties and ready accessibility. Thus, the ability to site-specially conjugate nearly any off-the-shelf IgG is an enabling technology that opens up a variety of applications and may allow the development of antibody conjugates to be “crowd-sourced” by researchers at large.

### Example 3: Formation of Bispecific Antibodies

To prepare bispecific antibodies, photoreactive antibody binding domains, e.g., Protein Z or Protein G adapters, can be modified with a linking module or a member of a binding pair that allows two antibody binding domains (i.e. adapters) to be linked together. There are many options for linking modules and they can generally be broken into three categories: biological linking modules, chemical linking modules, or oligonucleotides.

Biological linking modules can be fused in frame with the photoreactive AbBDs at the N- or C-terminus. Examples, of biological linking modules include SpyCatcher/SpyTag, split inteins, heterodimeric proteins that possess an affinity for each other (e.g., c-fos and c-jun, leucine zippers, peptide velcro, etc.), dock-and-lock proteins, sortase substrates, etc.

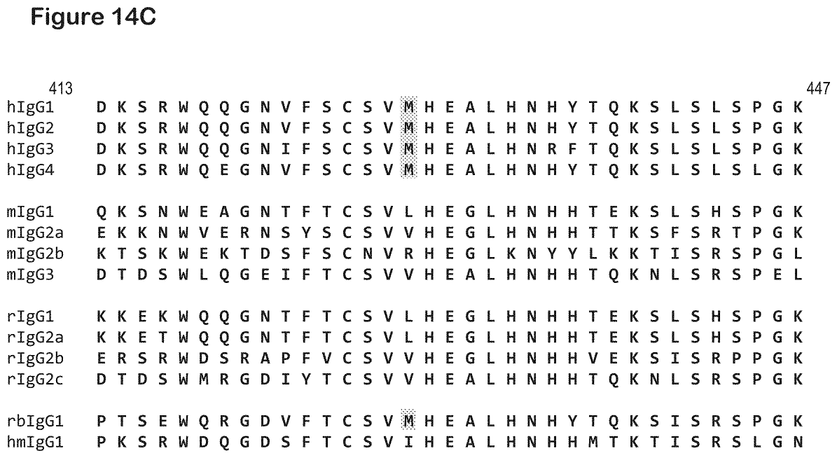

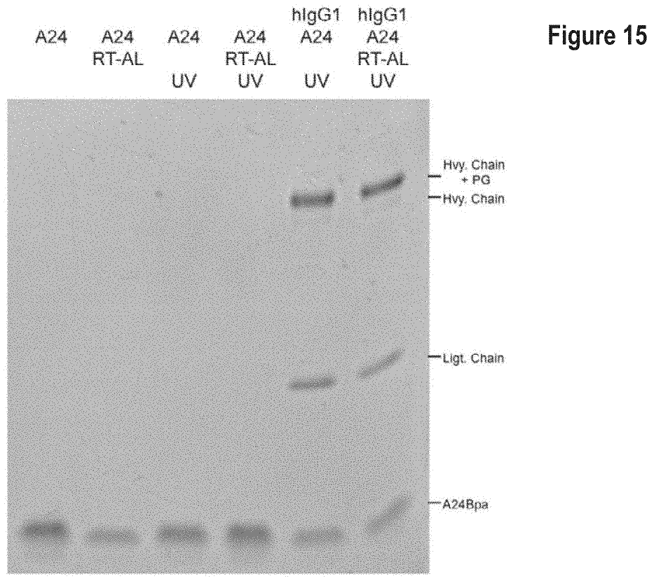

To demonstrate proof-of-principle, SpyTag (Mod.A) and SpyCatcher (Mod.B) were fused in frame at the C-terminal end of the Protein G adapters (FIG. 16). Note that with this approach, expressed protein ligation (e.g., STEPL) is not required to form bispecific antibodies, but could still be used to add additional functionality at the C-terminus of the Protein G adapter-SpyCatcher/SpyTag fusion protein (e.g., imaging agent, drug, etc.). Once the SpyTag and SpyCatcher fusion proteins were expressed and purified, they were photocrosslinked to cetuximab and OKT3 antibodies, respectively (FIG. 16, lanes 2 and 4). The unlabeled antibodies are shown in lanes 1 and 5, for comparison. Covalent linkage of the adapter protein resulted in a clear upward shift of the IgG band in the non-reducing gel and the heavy chain band in the reducing gel. The non-reducing gel confirmed that nearly all of the IgG was labeled with one or two adapters. Mixing of the two adapter-IgG conjugates resulted in the specific formation of bispecific antibodies (FIG. 16, lane 3; dimer). Some antibody monomers still exist as well as some higher order conjugates (e.g., trimers), but the predominant species are bispecific antibodies. Since, SpyCatcher forms a covalent linkage exclusively with SpyTag and not itself, and vice versa, the bispecific antibody that is formed is a heterodimer consisting of one cetuximab antibody and one OKT3 antibody. This was confirmed via western blotting (FIG. 17). Specifically, OKT3, cetuximab, and the bispecific antibody were run on non-reducing SDS-polyacrylamide gel. The OKT3 was probed using a anti-mouse secondary antibody labeled with IRdye800. The blot was then stripped, and subsequently labeled with an anti-human secondary antibody labeled with IRdye800. The OKT3 was only labeled with the anti-mouse secondary antibody. The cetuximab was only labeled with the anti-human secondary antibody. The bispecific antibody was labeled with both secondary antibodies.

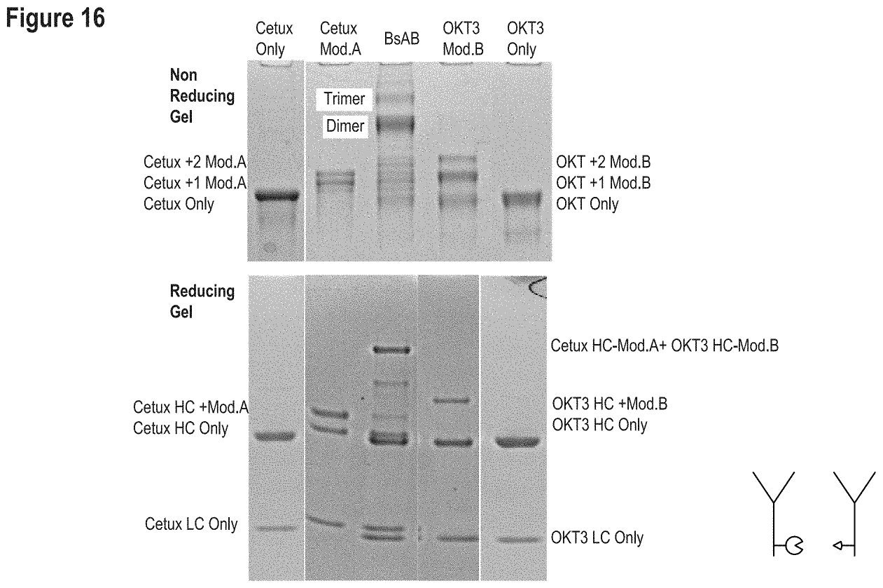

Bispecific antibody formation using SpyCatcher and SpyTag linking modules is efficient and stoichiometric (FIG. 18). Yield is >50%, of total inputting IgGs. It is also reproducible. Covalent modification of individual IgG with either SpyCatcher- or SpyTag-adapter proteins takes less than 120 min. Bispecific antibody formation is fast, nearly plateauing after 30 minutes. No increase in multimer formation is seen over time, which is likely due to unfavorable sterics effects.

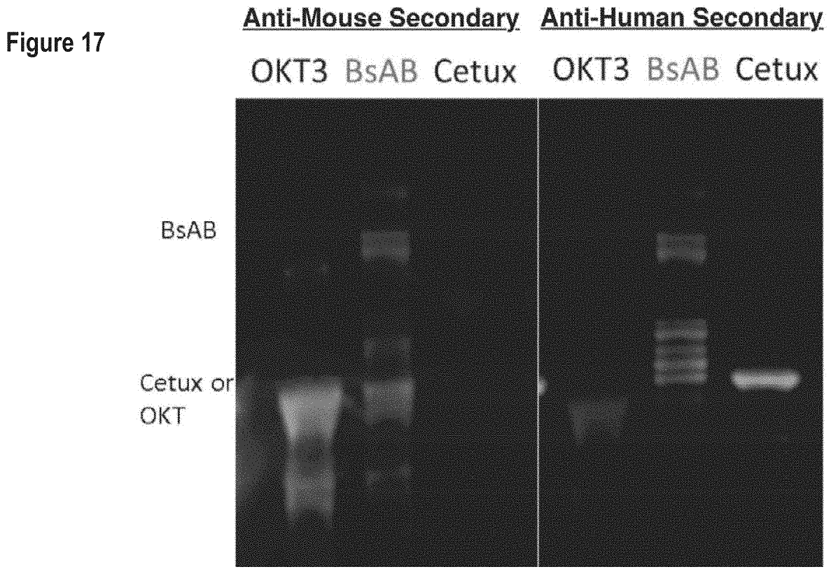

Highly pure bispecific antibody samples can be obtained by performing FPLC (FIG. 19) or other standard purification methods. Alternatively, highly pure samples can be obtained if IgG is only modified with an adapter protein on one of its heavy chains, since this prevents the formation of trimer and other higher order species.

As an alternative to biological linking modules, chemical linking modules can also be added to the photoreactive AbBDs at or near the N- or C-terminus. Examples, of chemical linking modules include azide/alkyne, azide/DBCO, tetrazine/TCO, aldehyde/oxyamine, etc. Click chemistry pairs are a favorable choice since they are bio-orthoganol and highly efficient, but other chemical linking modules known in the art can just as easily be used.

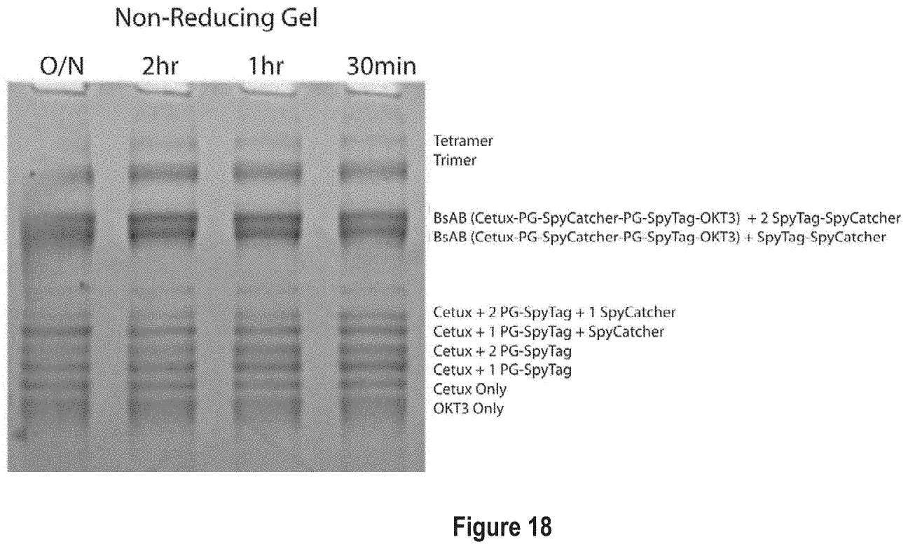

To demonstrate proof-of-principle, dibenzocyclooctyne (referred to as DBCO or ADIBO) and azide (N₃) labeled peptides were ligated to the C-terminal end of the Protein Z adapters via STEPL (FIG. 20). The ADIBO- and azide-labeled adapters were then both photocrosslinked to rituximab antibodies (FIG. 20, lanes 1 and 3). The ADIBO-IgG conjugate gives a red fluorescence signal on the gel since a TAMRA dye was also included on the peptide used in the STEPL reaction. The azide-IgG conjugates shows up as green since a FAM dye was included on the peptide used in the STEPL reaction. Mixing of the ADIBO-IgG conjugate with an azide-Protein Z adapter led to a clear shift in the ADIBO-IgG band, i.e. click product (FIG. 20, lane 2). Similarly, mixing of the azide-IgG conjugate with a ADIBO-Protein Z adapter led to a clear shift in the azide-IgG band, i.e. click product (FIG. 20, lane 4). Since, ADIBO forms a covalent linkage exclusively with azide and not itself, and vice versa, only the desired IgG-protein conjugates are formed. No homodimers are formed.

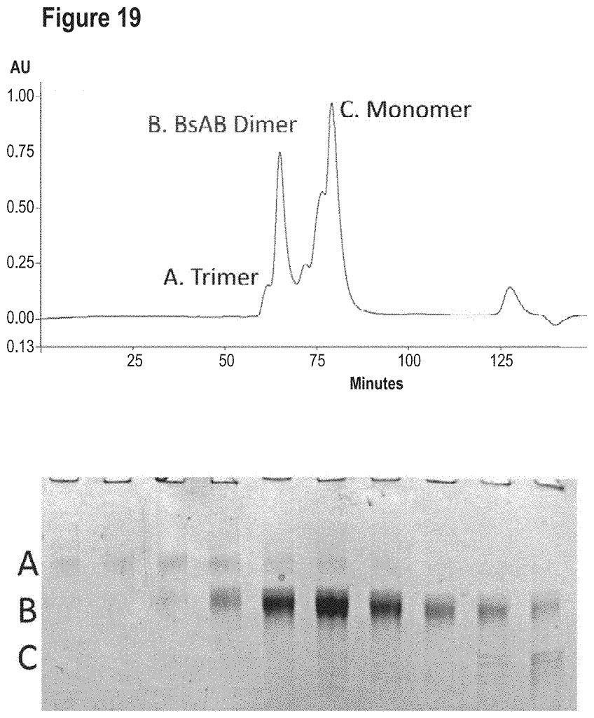

As a second example of using chemical linking modules to form bispecific antibodies, TCO and tetrazine labeled peptides were ligated to the C-terminal end of the Protein Z adapters via STEPL (FIG. 21). The TCO- and tetrazine-labeled adapters were then photocrosslinked to OKT3 and rituximab antibodies, respectively (FIG. 21, lanes 1 and 3). Mixing of the TCO-OKT3 conjugate with the tetrazine-rituximab conjugate resulted in the specific formation of bispecific antibodies (FIG. 21, lane 2). Some antibody monomers (i.e. heavy chain-Pz) still exist as well as some IgG-Protein Z-Protein Z conjugates (i.e. heavy chain-Pz clicked to free Pz). IgG-Protein Z-Protein Z conjugates resulted from the incomplete removal of azide and ADIBO-Protein Z adapters, which were never covalently linked to IgG.

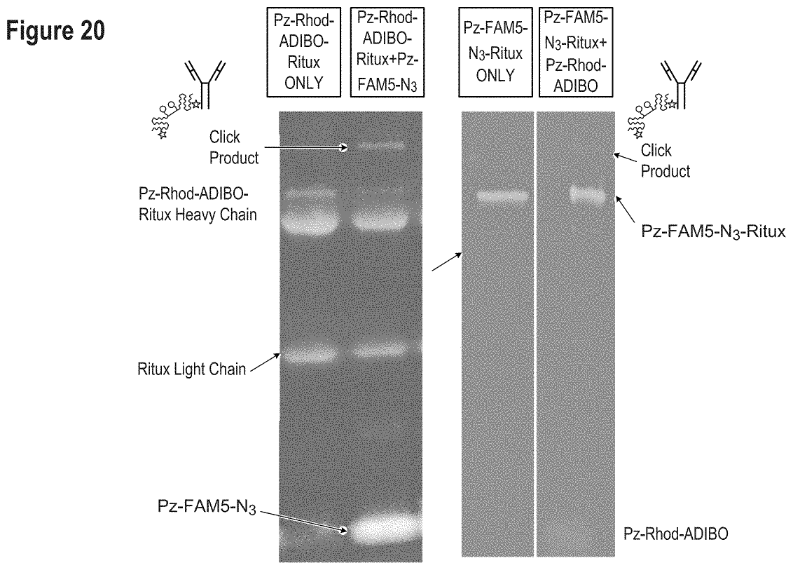

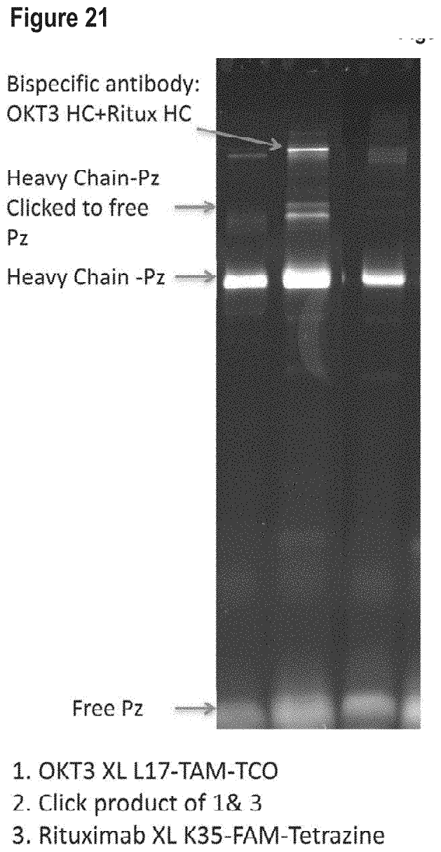

As an alternative to using linking modules to form bispecifics, it is also possible to express a single fusion protein containing two AbBDs. While it is very straightforward to produce homodimers using this type of fusion protein, it is also possible to produce heterodimers (i.e. bispecific antibodies) if each AbBD has unique specificity for a specific IgG subtype. This was demonstrated by fusing a Protein Z adapter protein with the BPA photocrosslinker located in the L17 position (Pz^{L17}) to a Protein G adapter with the BPA located in the K28 position (PG^{K28}). Pz^{L17} has unique specificity for mouse IgG2 as such as OKT3 while PG^{K28} has unique specificity for human IgGs such as cetuximab. As a result, an antibody dimer is only formed when both cetuximab and OKT3 are mixed with the Pz^{L1}7-PG^{K28} fusion protein (FIGS. 22 and 23, lanes 1-3). Addition of only a single antibody, cetuximab or OKT3, results in little to no dimer formation (FIGS. 22 and 23, lanes 4 and 5).

### Example 4: One-Step Production of Bispecific Antibodies

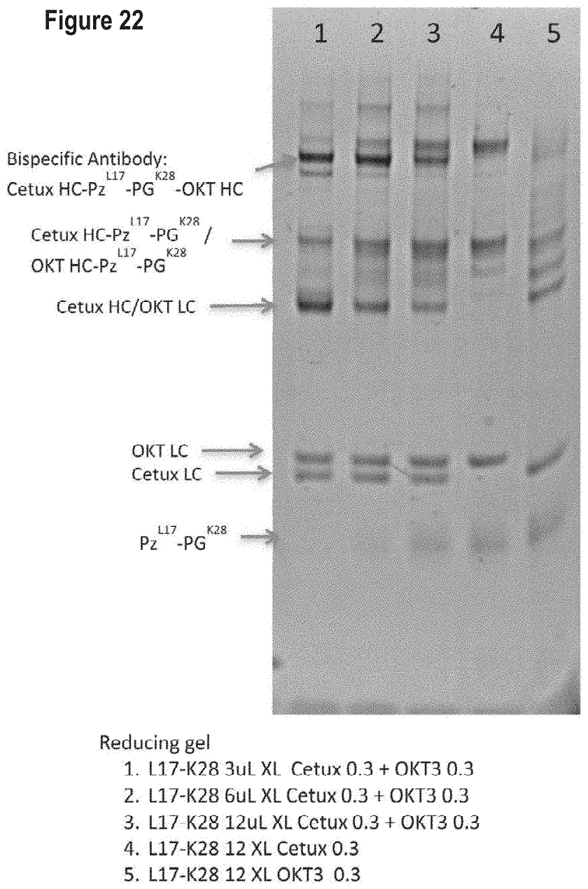

Recently, a rapid and site-specific bioconjugation technique was developed that allows for the attachment of an anti-CD3 scFv (or any other scFv) to any full-length human IgG. Our technique relies on a small antibody-binding domain (AbBD) that is engineered to contain a photoreactive unnatural amino acid (benzoyl-phenylalanine, BPA) in its Fc-binding site (FIG. 24). The AbBD used is based on small (˜6.5 kD), thermally stable domain of Protein G (HTB1). The introduction of a photoreactive amino acid allows for the formation of a covalent linkage between an scFv-AbBD fusion protein and IgG, to prevent dissociation in serum. The AbBD is capable of binding to both heavy chains of IgG (di-conjugated), thereby creating a tetravalent bispecific antibody. However, it is also possible to create a bispecific antibody with only a single scFv (i.e. trivalent/mono-conjugated). Both formats are tested. Some attributes of this approach are that it is simple, rapid (<2 hrs), efficient (˜100% of antibody is labeled), has no effect on antibody affinity, is amenable to high-throughput production, and either mono-conjugated or di-conjugated products are easily purified.

Mono-conjugated bispecific antibodies: According to previous reports, binding of Protein G to Fc sites of IgG does not prevent or sterically interfere with the attachment of these antibodies to the Fc receptor. Therefore, ADCC and CDC function is not expected to be lost with this bispecific antibody format. However, Protein G does prevent binding to the neonatal Fc receptor (FcRn). This leads to a half-life of 12 hours for IgG-protein G conjugates (non-crosslinked), which is much shorter than the half-life of 1 to 3 weeks for IgG. However, it is significantly longer than the half-lives of scFv's, which can be as short as 30 min.

In an attempt to maintain the long circulation half-life of native IgG, antibodies with only a single Protein G adaptor are prepared (FIG. 11), freeing up the adjacent heavy chain for FcRn binding. Mono-conjugated IgG may retain, at least partially, FcRn-mediated IgG recycling. It has been shown that FcRn binds to each site on IgG independently, with identical affinity. Circulation times can be determined. Having monovalency for CD3 is also expected to eliminate concerns over the level of cytokine release upon T cell binding. Di-conjugated bispecific antibodies are prepared and evaluated. Di-conjugated bispecific antibodies do offer the advantage of higher affinity for T cell targets and several tetravalent bispecific antibodies have entered clinical trials, including AbbVie/ABT-122, Sanofi/SAR156597, and Merrimak/MM141. The flexibility to create both mono-conjugated and di-conjugated bispecifics is a novel and valuable feature of this approach and could provide insight into designing optimal bispecific antibodies.

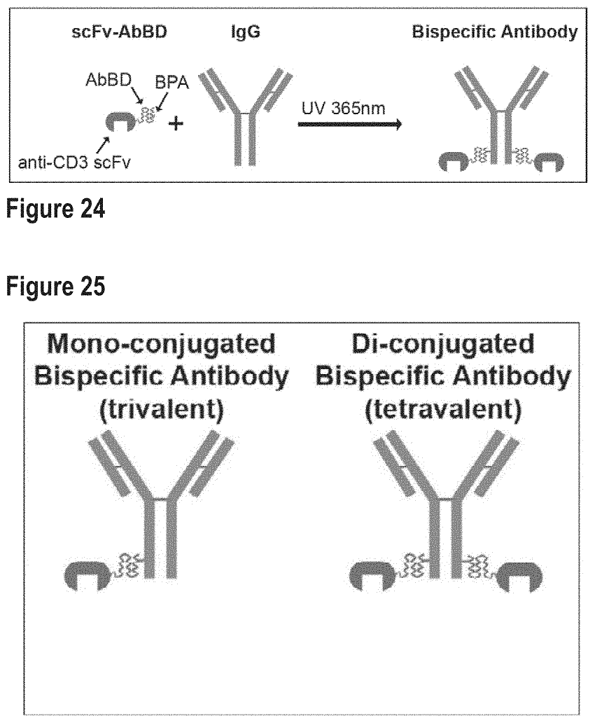

Sortase-Tag Expressed Protein Ligation (STEPL): Recently, a technique was developed that allows the c-terminus of any single chain protein to be labeled with nearly any desirable compound, including drugs, imaging agents, biomolecules, chemical handles, haptens, polymers, nanoparticles, etc. This technique relies on Sortase A (SrtA). SrtA is a calcium-assisted transpeptidase that is responsible for anchoring surface proteins to the peptidoglycan cell wall of Gram-positive bacteria. The enzyme cleaves the peptide bond between the amino acids T and G, within the motif, LPXTG (SEQ ID NO: 21). The products remain transiently attached to SrtA, until the N-terminal glycine of another protein displaces the C-terminal fragment and forms a new peptide bond between the two-peptide chains. To take advantage of this site-specific ligation reaction, a single fusion protein construct was created that contains LPXTG, (SEQ ID NO: 21), SrtA, and a His-tag, respectively, so that it can be fused to the C-terminal end of a desirable single chain protein (FIG. 26). This technique is utilized to label the anti-CD3 scFv and scFv-AbBD with a copper chelate, NOTA (1,4,7-triazacyclononane-N,N′,N″-trisacetic acid) for nuclear imaging. This allows for the facile creation of a companion diagnostic without adding any additional steps to the workflow.

One advantage of the bispecific antibody production method described here is that an “off”-the-shelf full-length antibody can be used with no need for protein engineering, cloning, or other modifications. This will make bispecifics more accessible to academic labs, allowing bispecifics to be tested in a wider range and more creative applications. Moreover, since bispecific antibody production is rapid (<2 hrs) and efficient (˜100%). This technique is amenable to high-throughput production, which is not currently possible with any other technique. This may allow for rapid screening of bispecific antibody pairs (e.g., different targets, different epitopes, different affinities) for optimal performance. Other advantages include the ability to swap between murine and human antibodies that target the same epitope and easily add additional functionality—toxins, imaging agents, drugs, radiopharmaceuticals or other chemical modifications—via STEPL. Notably, there are many issues that can ultimately influence the clinical applicability and utility of a bispecific antibody; however, even if this approach proves to be unfit for clinical use, it is expected that the ability to rapidly and easily screen antibodies for optimal performance could still be used to guide other bispecific antibody production techniques. This is expected to hold particularly true for other tetravalent bispecific antibody formats that closely resemble the bispecific antibodies that we are creating, e.g., IgG-scFv, scFv₂-Fc, DVD-Ig, etc.

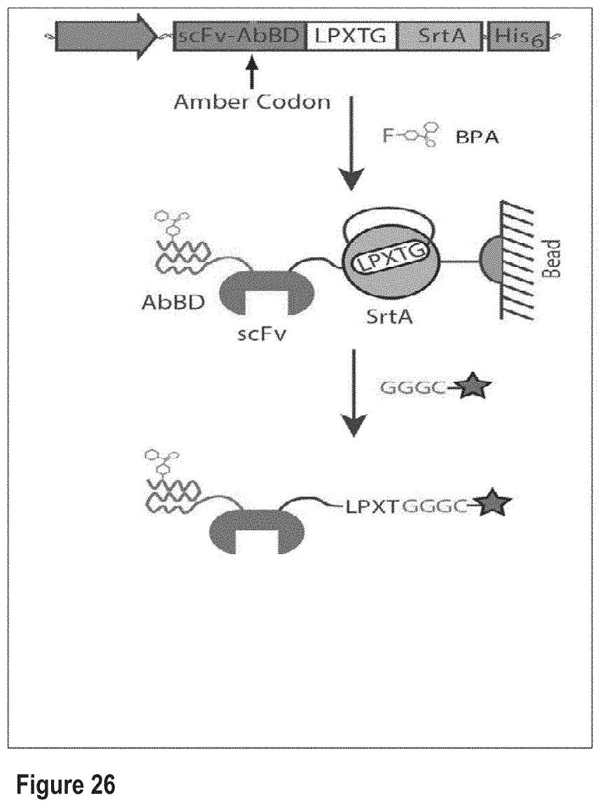

Formation of bispecific antibodies using AbBD-scFv fusion proteins: To prepare bispecific antibodies, a photoreactive AbBD was fused to an anti-CD3 scFv (OKT3 parent antibody). To create a bispecific antibody, the expressed AbBD-scFv is simply mixed with the IgG of choice and photocrosslinked for ≤2 hrs. To demonstrate the simplicity of the approach, 4 unique bispecific antibodies were created in parallel (FIG. 27). Because of the high crosslinking efficiency between the photoreactive AbBD and IgG, essentially just two species exist after the photoreaction, diconjugated IgG and free AbBD-scFv. This makes it extremely easy to obtain highly pure tetravalent bispecific antibody, since the free AbBD is easily removed using ultrafiltration spin columns (100 kDa MWCO, Millipore). If necessary, mono-conjugated IgG and unconjugated IgG can be removed using Protein A/G beads, since the AbBD sterically blocks the di-conjugated IgG from interacting with Protein A/G.

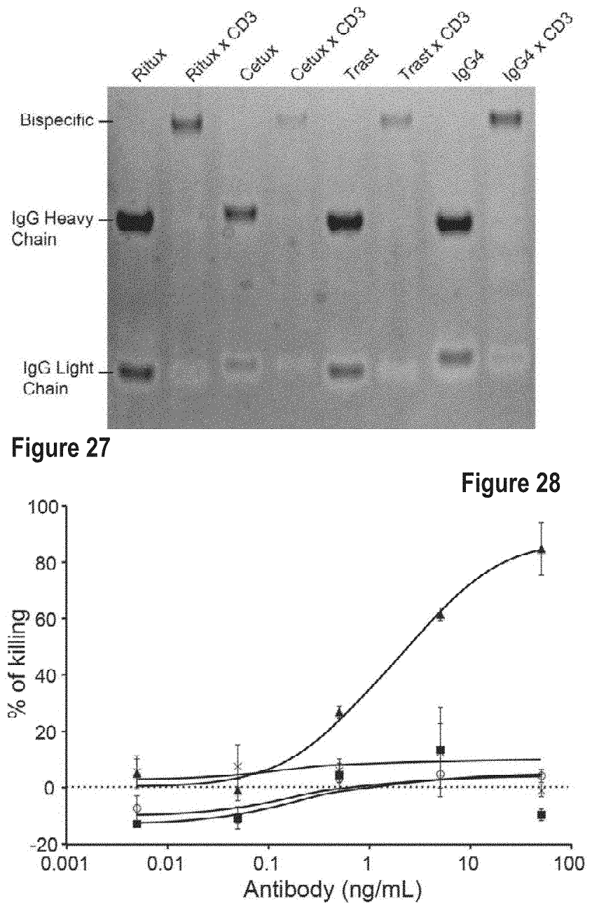

Confirmation of bispecific antibody functionality: To demonstrate that the (di-conjugated) bispecific antibodies created using our one-step photoreaction were able to mediate cell killing, T cell-mediated cell lysis assay was performed (FIG. 28). Specifically, bispecific antibodies composed of rituximab and anti-CD3 scFv's were incubated with CD20-positive Jeko B cells. PBMCs were added at an effector-to-target ratio of 10:1 and incubated for 24 hrs. Cytotoxicity was measured via a chromium release assay. The bispecific antibody exhibited a dose-dependent cytotoxic effect with statistically significant cytotoxicity (27% lysis) measured at 0.5 ng/mL and an EC₅₀ of ˜2 ng/mL. This is very similar to what others have observed with anti-CD20/CD3 bispecific antibodies, although direct comparisons are difficult due to cell line-to-cell line and PBMC donor-to-donor variability.

The potency of this construct is further improved by testing an alternative anti-CD3 scFv (UCHT1 parent antibody) and by varying the length of the linker between the AbBD and the scFv. Notably, no cytotoxicity was observed with CD20 negative K562 cells. Moreover, no toxicity was observed when rituximab or a mixture of anti-CD3 scFv and rituximab were incubated with Jeko B cells in the presence of PBMCs at a 10:1 effector-to-target ratio.

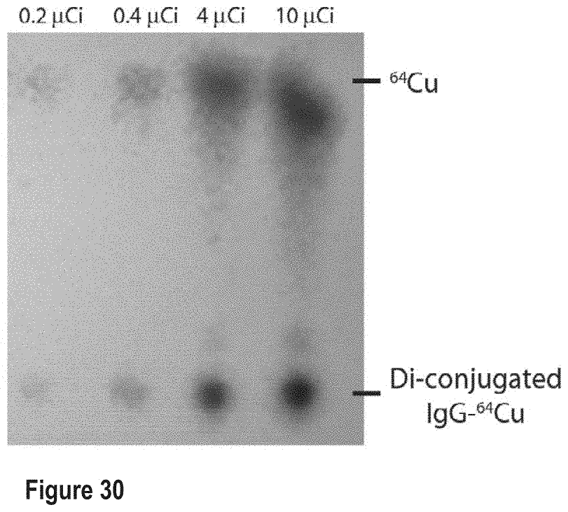

STEPL is utilized to label the anti-CD3 scFv and scFv-AbBD with a copper chelate, e.g., NOTA, for nuclear imaging. This allows for the facile creation of a companion diagnostic without adding any additional steps to the workflow. Data was acquired showing efficient labeling of di-conjugated IgG with Cu-64 (FIG. 30). Notably, if no additional labels are desired at the C-terminus of the AbBD-scFv, triglycine can simply be used to catalyze release from the affinity column.

### Example 5: Rapid Production of Bispecific Antibodies from Off-the-Shelf IgGs with High Yield and Purity

Bispecific antibodies (BsAb) refer to a class of biomacromolecules that are capable of binding two antigens or epitopes simultaneously. This can elicit unique biological effects that cannot be achieved with either individual antibody or with two unlinked antibodies. Bispecific antibodies have been used for targeting effector cells to tumor cells, preferential targeting of cells expressing two target biomarkers over cells expressing either target biomarker individually, or to couple two molecular targets on the same cell surface to trigger unique intracellular signaling pathways. In this Example, two related methods are presented that allow direct, rapid assembly of bispecific antibodies from any two “off-the-shelf” Immunoglobulin G (IgG) antibodies, in as little as one day. Both workflows can be summarized into two steps: 1) attach a small photoreactive antibody binding domain (pAbBD) fused to SpyCatcher or SpyTag (peptide-protein partners derived from the S. pyrogenes fibronectin-binding protein FbaB) to each component IgG, respectively; 2) assemble the BsAb through the spontaneous isopeptide bond formation that occurs between SpyTag and SpyCatcher. These approaches allow production of BsAbs from any two IgG molecules without the need to elucidate their amino acid sequences or genetically alter their structure. Binding assays and T cell-mediated cytolysis assays were performed to validate the binding and functional properties of Trastuzumab×Cetuximab BsAb and Cetuximab×OKT3 BsAb, respectively. This approach allows rapid, low-cost production of highly homogenous tetravalent BsAbs in a modular fashion, presenting an opportunity to quickly evaluate antibody pairs in a BsAb format for unique or synergistic functionalities.

### Introduction

Bispecific antibodies (BsAb) refer to a class of biomacromolecules that consist of two unique targeting domains that can simultaneously bind two different epitopes or antigens. The ability to bind to two different targets simultaneously can confer novel functionality to BsAbs. For example, BsAbs can bridge effector cells and target cells. In one such case, bispecific T cell engagers (BiTEs), which contain tumor surface antigen-binding domains and T cell-specific CD3-binding domains, recruit T-cells to promote cytolysis of tumor cells. BsAbs can also be used to preferentially bind cells that simultaneously express two molecular targets, compared to cells that express only one, due to increased avidity. The ability of BsAbs to bind two different epitopes on a cell surface simultaneously can also interfere with signaling pathways in unique ways that is not possible with either antibody alone or the combination of unlinked antibodies. The crosslinking of cell-surface antigens can trigger, redirect, or inhibit cross-talk between signaling pathways. The simultaneous binding of two receptors is especially valuable in angiogenesis inhibition and tumor treatments, where the biological signals are often redundant and target cells can evade inhibition of one receptor by upregulating another. With several bispecific antibodies, such as emicizumab and blinatumomab, gaining FDA approval and dozens more in clinical trials, BsAbs are garnering growing interest as a therapeutic modality for treating a wide range of diseases such as cancer, hematological disorders, inflammatory disorders, diabetes, and Alzheimer's disease.

As of 2019, more than 100 BsAb formats have been developed. Structurally, BsAbs are often conceptualized as an assembly of two antigen binding moieties, sometimes with the addition of crystallizable fragments (Fc) and linkers. Together, these features affect the level of production complexity, stability, functional valency, mechanism of action, plasma half-life, and toxicity of BsAb therapeutics. Many different approaches have been employed to generate bispecific antibodies, including chemical conjugation methods, the fusion of antibody fragments (e.g., BiTEs), and genetic engineering of full-length antibodies that allow controlled pairing of two antibodies along their line of symmetry, e.g., “knob-into-hole”. While each of these approaches can yield functional BsAbs, chemical methods are generally inefficient and/or result in highly heterogeneous products, limiting their adoption, and genetic methods generally require several rounds of recombinant protein engineering in addition to construction and optimization of mammalian cell lines. The time and cost associated with generating such genetically engineered BsAbs is often only justified with strong evidence supporting the proposed dual-targeting strategy. Therefore, while these platforms are useful in targeting well-studied or predictable biological pathways, there is still a need for an accessible technology that allows the rapid testing of any two antibody pairs for unique or synergistic activity. This is perhaps most easily achieved if BsAbs can be assembled from any pair of “off-the-shelf” Immunoglobulin Gs (IgGs).

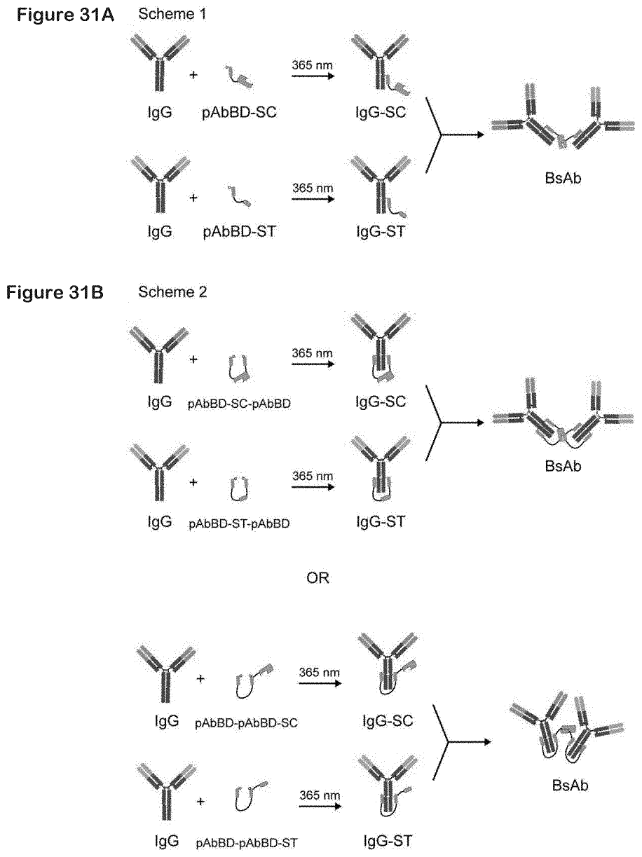

In this Example, two methods are presented for rapid, in vitro assembly of tetravalent, bispecific antibodies from two full-length IgGs. Both methods use small photoreactive antibody binding domains (pAbBDs) fused to SpyCatcher (SC) or SpyTag (ST) to steer full-length IgG molecules into forming heterodimers (FIG. 31). pAbBDs are composed of IgG-binding domains derived from the bacterial protein G or protein Z and allow highly-efficient photocrosslinking to the heavy chains of nearly any IgG, from a diverse range of hosts and subclasses. pAbBDs can be expressed as a fusion protein in series with other proteins of interest to label IgGs covalently and in a site-specific manner. Here, pAbBDs are used to label IgGs with either SpyCatcher or Spytag. This protein-peptide binding pair was originally developed from the S. pyrogenes fibronectin binding protein FbaB domain and can spontaneously form a covalent isopeptide bond. Therefore, once SpyCatcher and SpyTag are covalently attached to two different antibodies via the pAbBDs, they can drive the formation of a stable BsAb. This eliminates the need for genetic engineering of the component antibodies and allows the use of an off-the shelf IgG to create BsAbs in a modular fashion. Thus, this represents a powerful new approach to test any combination of antibodies in a BsAb format for unique or synergistic functionalities.

### Methods

### Cloning, Expression, and Protein Purification

All pAbBD fusion proteins are expressed and purified via the Sortase-Tag Expressed Protein Ligation (STEPL) method. The pSTEPL plasmid was first modified to include a His₁₂ tag (SEQ ID NO: 22) instead of a His₆ tag (SEQ ID NO: 23). Plasmids encoding variants of pAbBD-SpyCatcher/SpyTag and pAbBD-SpyCatcher/SpyTag-pAbBD were cloned via InFusion (Takara Bio) reactions of their respective GeneBlock (IDT) into pSTEPL-His₁₂ plasmids (“His12” is disclosed as SEQ ID NO: 23). The first generation SpyCatcher (ATHIKFSKRDEDGKELAGATMELRDSSGKTISTWISDGQVKDFYLYPGKYTFVETAA PDGYEVATAITFTVNEQGQVTVN; SEQ ID NO: 17) and SpyTag (AHIVMVDAYKPTK; SEQ ID NO: 18) constructs were used here. For pAbBD-SC/ST, a (GGS)₇ (SEQ ID NO: 24) linker is used to connect the pAbBD with SC or ST. For pAbBD-SC/ST-pAbBD, the linker on the N-terminus of SC/ST is (GGS)₇ (SEQ ID NO: 24). For the linker following SC/ST, two versions with either (GGS)₄ (SEQ ID NO: 25) and (GGS)₇ (SEQ ID NO: 24) were produced. Sequences are verified via restriction digest and Sanger sequencing.

Plasmids were then co-transformed into T7 Express Competent E. coli cells (New England Biolabs) with pEVOL-pBpF plasmid (Addgene). The cells were spread onto a Luria Broth (LB) agar plate containing ampicillin (100 μg/mL) and chloramphenicol (20 μg/mL) incubated at 37° C. After 16-18 hours, a starter culture is prepared by inoculating bacterial colonies in 3 mL lysogeny broth (LB) supplemented with ampicillin (100 μg/mL) and chloramphenicol (20 μg/mL) and cultured for 16 hours in a shaker (225 rpm) at 37° C. For expression, the starter cultures were inoculated into Auto Induction Media LB Broth (Formedium) containing glycerol (0.6% v/v), chloramphenicol (20 mg/L), ampicillin (100 mg/L), L-benzoylphenylalanine (BPA, 200 μM, Bachem) and L-arabinose (0.1% w/v) at a 1:1000 ratio. The bacterial culture was then allowed to grow on shaker (225 rpm) at 37° C. for 18 hours.

Cultures were pelleted down, resuspended in 1% OTG in PBS (w/v), and lysed on rotation at 25° C. for 30 min. The lysate was then briefly sonicated on ice and centrifuged at 15,000 g for 20 min at 4° C. The resulting supernatant was transferred into in a 10 mL Poly-Prep chromatography column (Bio-Rad) and co-incubated with TALON® Superflow Metal Affinity Resin (Takara Bio USA) at room temperature on rotation for 30 min. The resin was then washed with 1 column volume (CV) PBS, 1 CV of 20 mM imidazole in PBS, and 2CV PBS. The resin was then incubated and eluted in 2 mM triglycine and 125 μM CaCl₂) in PBS for an hour at 37° C.

The eluted proteins were concentrated using a 3K MWCO Amicon Ultra Centrifugal Filter (Millipore) and purified via size-exclusion chromatography on a Superdex s30 10/300L column (Cytiva). Total protein concentration was measured with Pierce™ BCA Protein Assay Kit (ThermoFisher) and protein size was verified via SDS-PAGE. Protein gels are stained with SimplyBlue Safe Stain (ThermoFisher). Protein sizes were compared against Novex Sharp Prestained Protein Standards (ThermoFisher). Protein samples were aliquoted, frozen, and stored at −80° C.

Crosslinking pAbBD Fusion Proteins to IgGs

pAbBD fusion proteins were added to IgGs and the mixture was exposed to 365 nm UV light in ice bath using a UVP CL-1000L UV crosslinker, which houses 5×8 watt bulbs. For bispecific antibody production, the crosslinking reactions were set up at an IgG concentration of 0.05-0.3 mg/mL, using a pAbBD-fusion-to-IgG ratio of 1:1 to 32:1 and an irradiation time of 2 hours. The crosslinking mixture was then concentrated with a 50K MWCO Amicon Ultra Centrifugal Filter (Millipore) and purified via size-exclusion chromatography on a Superdex s200 increase 10/300L column (Cytiva). Crosslinking product was analyzed with SDS-PAGE and ImageJ. Total protein concentrations were measured via BCA assays.

### Purification of Crosslinked IgG Using Recombinant Protein G Agarose

IgG crosslinked to pAbBD-SpyCatcher/SpyTag was allowed to bind recombinant protein G resin for 30 min at room temperature on rotation. The resin was then spun down, washed three times with 50 mM Tris-HCl with 150 mM NaCl. 0.1 M Glycine-HCl (pH=2.7) was added to elute bound protein from spin columns into 1 M Tris-HCl (pH=8), which neutralizes the solution.

### Production of BsAb Using Isopeptide Bond Formation

For isopeptide bond formation between SpyCatcher and SpyTag, an equimolar mixture of IgG-SC and IgG-ST in PBS, each at 1 μM (0.17 mg/mL) was incubated at 4° C. overnight. Bispecific antibodies were purified via size-exclusion chromatography on a Superdex s200 increase 10/300L column (Cytiva). When necessary, BsAbs were concentrated using a 50K MWCO Amicon Ultra Centrifugal Filter (Millipore).

### Fixed-Cell Surface Ligand Binding Assay

MDA-MB-468 (EGFR⁺, HER2⁻) and T6-17 (HER2⁻, EGFR⁺) were each assayed for their respective binding to Cetuximab, Trastuzumab and Trastuzumab×Cetuximab BsAb. For each cell line, cells were seeded in black-wall, transparent-bottom 96-well plates (Corning) at 5,000 cells/well. The cells were incubated at 37° C. in a 5% CO₂ humidified incubator for 3 days or until 90% confluency. Cells were then fixed in neutral buffered formalin solution (Sigma-Aldrich) for 15 min at 25° C. and washed three times with 0.05% PBST. Plates were stored at 4° C. with 100 μL 0.05% PBST in each well and used within 2 weeks of production.

In each binding assay, cells were first blocked with 10% Normal Goat Serum (Life Technologies) for 15 minutes at 25° C. and washed three times with 0.05% PBST. Antibodies and bispecific antibodies in blocking buffer (lx PBS with 0.05% Tween 20 and 0.25% BSA) were added in duplicates to cells in a three-fold serial dilution from 100 nM to 0.033 nM and allowed to bind at 25° C. for an hour on a horizontal shaker. Cells were then washed three times with 0.05% PBST to remove unbound antibodies. Cells were then incubated in 1:1000 dilution of Goat Anti-Human IgG Fc Secondary Antibody conjugated to PE (eBiosciences) for an hour at 25° C. Cells were washed again with 0.05% PBST three times before measuring fluorescence at 544/585 nm in 0.05% PBST with a Tecan M200 Infinite plate reader.

Duplicate untreated cells remain in 0.05% PBST through binding and washing steps but are detected with the same method. Signals from these untreated cells were designated as background signal. Background signal was subtracted from all raw signals. Signals were then normalized, with 0% designated as the average minimal signals from each cell line and 100% the average maximal signals from each cell line. Normalized data were analyzed and plotted with GraphPad Prism8, both for original data and averaged data. Multiple unpaired t-tests were conducted for all pairings of EC50s (unaveraged) assuming Gaussian distribution (p<0.05).

### T Cell-Mediated Cytolysis Assay

MDA-MB-468 cells were maintained in DMEM (Corning) supplemented with 10% FBS (Corning) and 1% penicillin/streptomycin (ThermoFisher). Healthy human T cells were obtained from the Human Immunology Core (University of Pennsylvania) and expanded as previously described. Briefly, CD4 and CD8 T cells were incubated 1:1 and stimulated with CD3/CD28 Dynabeads (Gibco). Human IL-2 (Gibco) was maintained at a concentration of 50 IU/mL for 10 days. The Dynabeads were removed after 7 days of culturing, and the cells were maintained at 0.5-1 M/mL an additional 7 days. The cells were frozen down using a 1:1 mixture of X-VIVO media (Lonza) and 10% DMSO in FBS and allowed to rest in RPMI media (Corning) supplemented with 10% FBS and 1% penicillin/streptomycin 24 hours before cytolysis assays.

10,000 tumor cells were seeded per well 24 hours prior to adding BsAb treatments and T cells at an E:T ratio of 10:1. Controls included 3-fold serial dilutions starting at 100 nM or 10 nM of the monoclonal antibodies alone (Cetuximab and OKT3 separately) and an equimolar mixture of Cetuximab and OKT3. Tumor cytolysis was tracked up to 72 hrs post-treatment using xCelligence Real-Time Cell Analysis (ACEA Biosciences). Data were analyzed and plotted with GraphPad Prism8.

### Results

BsAb Production Using pAbBD-SpyCatcher and pAbBD-SpyTag (Scheme 1)

The initial approach that was tested to rapidly create bispecific antibodies involved first crosslinking two IgGs to either pAbBD-SC or pAbBD-ST, respectively, and then using the interaction between SpyCatcher and SpyTag to form a bridge between these two antibodies (FIG. 31A, Scheme 1). As proof of principle, bispecific antibodies were formed from different combinations of the clinical IgG antibodies, Trastuzumab (anti-Her2/neu), Cetuximab (anti-EGFR), and OKT3 (anti-CD3).

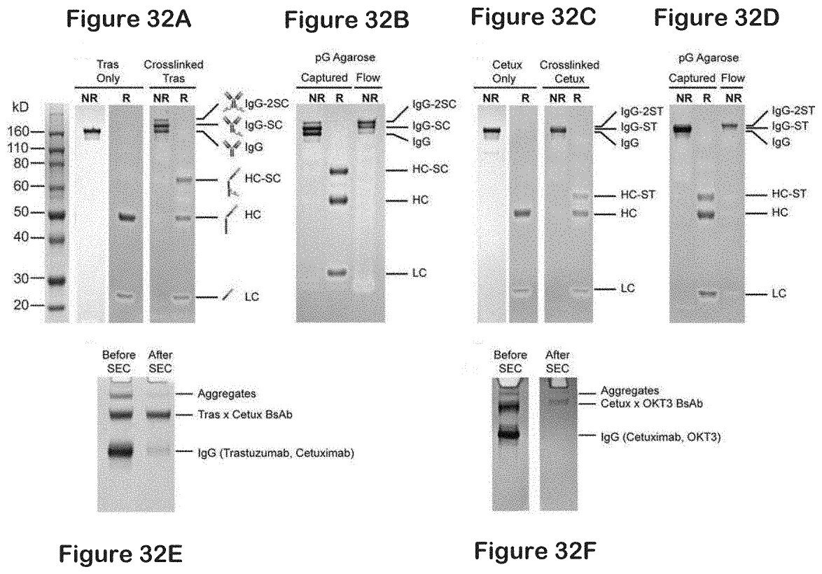

Since every IgG molecule has two heavy chains to which pAbBD can crosslink, Scheme 1 yields a heterogenous mixture consisting of uncrosslinked IgG, IgG conjugated to a single pAbBD-SC/ST, and IgG conjugated to two pAbBD-SC/ST, in addition to any unbound pAbBD-SC/ST. On non-reducing SDS-PAGE, this mixture shows up as 3 bands around 150 kDa, representing IgG crosslinked to two, one, and no pAbBD-SC/ST from top down. When reduced, this mixture contains the heavy chain crosslinked to one pAbBD-SC/ST (˜60-70 kDa), unlabeled heavy chain (˜50 kDa), and unlabeled light chain (˜25 kDa) (FIGS. 32A, 32B). While unconjugated IgGs will not be crosslinked in the SpyCatcher-SpyTag binding step and can be easily separated from 330 kDa BsAbs with size-exclusion methods, it is imperative to minimize the amount of IgG crosslinked with pAbBD-SC/ST on both heavy chains as it can form chains of 3 or more antibodies and can significantly reduce the yield of the correct BsAb product.

To maximize the relative amount of IgG conjugated to a single pAbBD-SC/ST, the amount of pAbBD-SC/ST was titrated against a fixed amount of IgG. The maximum percentage of IgG-pAbBD-SC/ST achieved was ˜40%, at a pAbBD to IgG ratio ˜1.2:1 (FIGS. 32A, 32C). To further enrich the population of IgGs labeled with a single SpyCatcher or SpyTag, recombinant protein G (pG) Agarose was used. The envisioned pG recovery process relies on the fact that pG Agarose binds IgG at the C_{H}2-C_{H}3 junctions, thus it is unable to bind IgGs crosslinked to two pAbBD-SC/ST. As a result, these constructs are ideally removed in the flow-through during the capture/wash steps. As shown in FIGS. 32B, 32D, however, pG Agarose recovery of IgG conjugated to one or no pAbBD proved to be less effective than expected as it also bound to dual-conjugated IgGs, even at a much lower pG Agarose-to-IgG ratio than recommended. This is hypothesized to be because pG Agarose can also bind to the Fab region. IgG-SC/ST was also found in the flow-through and was not captured as efficiently as unlabeled IgG. As a result, the pG Agarose recovery step only led to a slight enrichment of IgGs crosslinked to one or no pAbBD-SC/ST.

After pG Agarose recovery, IgG-SC and IgG-ST were incubated at equimolar concentrations at 4° C. overnight. BsAbs were formed with an overall efficiency of ˜20-40%. The unreacted IgGs and aggregates were removed via size exclusion chromatography (SEC), leading to a final purity of >90% (FIG. 32C, 32D) and an overall yield of ˜15%. While Scheme 1 led to the successful creation of BsAb, a notable drawback of this approach was the low efficiency of labeling IgG with a single pAbBD-SC/ST and the low percent of BsAbs recovered after SC-ST complementation. This prompted the invention of scheme 2.

BsAb Production Using pAbBD-SC-pAbBD and pAbBD-ST-pAbBD (Scheme 2)

To overcome the limitations of generating bispecific antibodies with pAbBD-SC/ST, a fusion protein was designed containing SpyCatcher or SpyTag flanked by two pAbBDs (FIG. 31B, Scheme 2). This method was designed to maximize the chance of crosslinking only one SpyCatcher or SpyTag per IgG by simultaneously occupying both C_{H}2-C_{H}3 junctions with a single construct.

Notably, a potential side-product of Scheme 2 is a homodimer, where two IgG molecules are crosslinked via a single pAbBD-SC-pAbBD or pAbBD-ST-pAbBD construct.

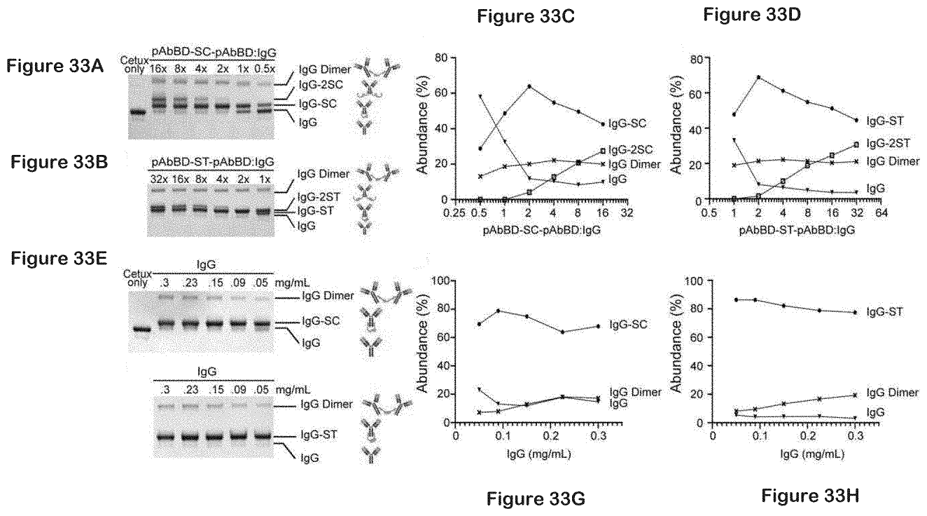

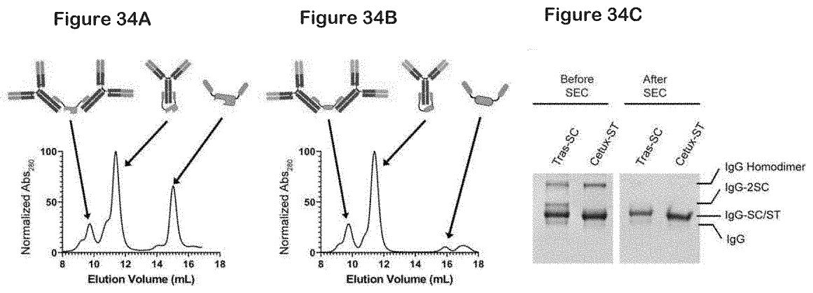

To maximize the relative amount of IgG that has been crosslinked by a single pAbBD-SC/ST-pAbBD, the molar ratio of pAbBD-SC/ST-pAbBD to IgG and the IgG concentration were varied (FIG. 33). The optimal crosslinking conditions were found to use ˜2 equivalent(s) of pAbBD fusion per IgG. IgG concentrations up to 0.3 mg/mL could be utilized. Under these conditions, approximately 70-80% of the IgG was labeled with just one SpyCatcher/SpyTag. For all experiments, photocrosslinking was performed for 2 hours at 4° C. The large size difference between IgG homodimers (˜300 kDa) and the desired product, i.e. IgG modified with a single SpyCatcher/SpyTag (˜170 kDa), allowed purification using size-exclusion chromatography (SEC) (FIG. 34).

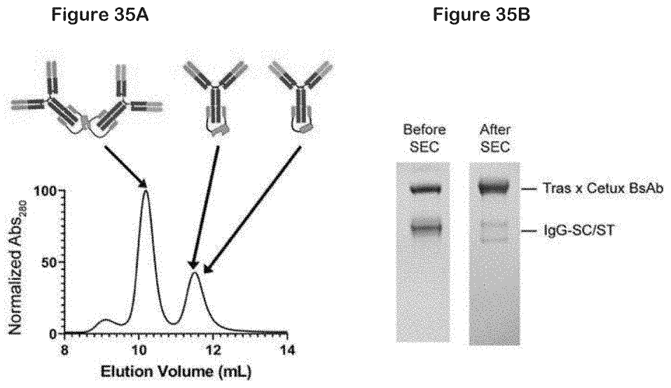

After successful isolation of IgG-SC and IgG-ST via SEC, the two conjugates were mixed at equimolar concentrations and incubated at 4° C. overnight. BsAbs were formed with an overall SpyCatcher/SpyTag reaction efficiency of ˜70%. The unreacted component IgGs were then removed via SEC, leading to a final purity of 90-95% (FIG. 35) and an overall yield of 40-50%. With Scheme 2, both the efficiency of labeling IgG with a single SpyCatcher/SpyTag and the efficiency of BsAb formation, owing to SC-ST complementation, was ˜2-times more efficient than Scheme 1 and the overall yield was >3-times higher, with a higher level of purity. Therefore, all subsequent studies were performed using the BsAbs generated using Scheme 2.

### Cell Surface Ligand Binding Assay

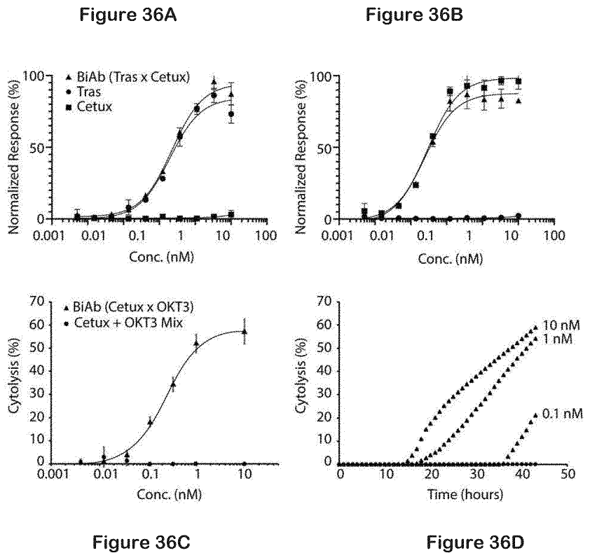

Binding assays were performed to survey the binding potential of Trastuzumab×Cetuximab BsAbs (anti-Her2×anti-EGFR) against HER2/neu- and EGFR-positive cells. BsAb binding was compared to that of monospecific IgGs from which the BsAbs were derived. T6-17 human breast cancer (HER2+/EGFR⁻) and MDA-MB-468 human breast cancer cell lines (HER2⁻/EGFR⁺) were used to evaluate binding to HER2 and EGFR, respectively. The BsAbs exhibited similar dose-dependent binding to both T6-17 and MDA-MB-468 cells as the respective individual parental IgGs (FIGS. 36A, 36B). Paired t-tests revealed no significant differences.

### T Cell-Mediated Cytolysis Assay

Cetuximab×OKT3 BsAbs (anti-EGFR×anti-CD3) were tested for their ability to induce T cell-mediated cytolysis of EGFR⁺ MDA-MB-468 tumor cells. When Cetuximab×OKT3 BsAbs were incubated with target cells in the presence of human T cells expanded in vitro, dose-dependent cytolysis was observed (FIGS. 36C, 36D). In comparison, an equimolar mixture of Cetuximab and OKT3 exhibited no cytolysis, in the presence of T cells, at equivalent doses and incubation time. A 10:1 effector-to-target ratio was used in all studies. These results confirm that cytolysis is mediated by the direct linkage between Cetuximab and OKT3. The BsAbs were effectively able to recruit T cells and allow formation of functional immunological synapses between tumor cells and T cells.

### Discussion

In recent years, there has been a clear interest in developing new methodologies to rapidly prepare bispecific antibodies; however, prior attempts that rely on chemical conjugations result in inefficient reactions, low yields, and aggregates. Under optimal conditions, the yield of purified BsAbs using traditional chemical conjugation techniques has been reported to be only as high as 15%, and that is with a final purity of just ˜70%. Genetic engineering approaches produce BsAbs with significantly higher homogeneity and purity (>95%), but it can take weeks or longer to acquire a functional product for testing, owing to complexities associated with expression, folding, and yield. Prior knowledge of the antibody sequence is also required in most cases, particularly if antibodies capable of binding specific targets are to be incorporated into the BsAb design. This presents a barrier to exploring and discovering new and unique antibody pairs that may exhibit interesting synergistic functionality.

To accelerate BsAb production, photoreactive antibody binding domains that site-specifically label nearly any off-the-shelf IgG with SpyCatcher/SpyTag was leveraged. The specific interaction between SpyCatcher and SpyTag was then used to drive the formation of a covalent isopeptide bond between the labeled IgGs. This allowed BsAbs to be generated against any two desired targets in less than a day, with high purity and yield. In contrast to genetic engineering approaches, this method does not require prior sequencing of the parental antibodies. Moreover, no recombinant protein engineering of the component IgGs is required and no mammalian/bacterial expression systems are required beyond the production of the pAbBD fusion proteins, which can be prepared in bulk. Two antibodies of choice are simply conjugated with SpyCatcher and SpyTag, respectively, and then combined to make a BsAb. The pAbBD is broadly compatible with antibodies from a wide range of hosts and subclasses, including all mouse, human, and rabbit isotypes. This feature makes this approach highly modular and could allow small panels of BsAbs to be rapidly generated by mixing and matching antibodies that have first been functionalized with SpyCatcher and/or SpyTag.

Between Scheme 1 and Scheme 2, Scheme 2 was found to be superior both in terms of the IgG labeling efficiency with a single SpyCatcher/SpyTag and the efficiency of BsAb formation. While scheme 1 produces 30-40% of IgG labeled with a single SpyCatcher/SpyTag, the overall yield of final BsAb product is only ˜15%, with a purity ˜90%. The biggest challenge associated with Scheme 1 was the undesirable formation of IgG modified with two SpyCatcher/SpyTag. This byproduct led to multimeric species composed of more than two IgG molecules (>450 kD) when SpyCatcher- and SpyTag-labeled IgG were combined. These multimeric species were difficult to avoid without significantly reducing the ratio of SpyCatcher/SpyTag-to-IgG, which leads to a large fraction of unlabeled IgG, with no improvement in the yield of IgG-SC/ST. This led us to explore various approaches to minimize the amount of IgG labeled with two SpyCatcher/SpyTag.

Size exclusion chromatography could not be used to separate IgG labeled with two SpyCatcher/SpyTag from IgG labeled with one SpyCatcher/SpyTag, due to the small size difference. As an alternative approach, Protein G resin was used to isolate IgG with only a single SpyCatcher/SpyTag, since one heavy chain is still available for capture. Unlabeled IgG is also captured, but it is not able to form BsAbs and can be easily separated from BsAbs on size exclusion chromatography. Since both heavy chains of IgG are blocked when labeled with two pAbBD-SC/ST, we expected that this construct would not be captured by the Protein G resin. However, while a capture step with Protein G resin did lead to some enrichment, there were still significant levels of the double-labeled IgG impurity. This is believed to be because Protein G has some affinity for the Fab domain. Perhaps, using an affinity resin that is specific for just the heavy chain could avoid this issue, but this approach was not tested here.

The use of pAbBD-SC/ST-pAbBD overcomes the shortcomings of using pAbBD-SC/ST since the two tethered pAbBD are much more likely to bind the same IgG than two different IgG, due to the proximity effect. This provides a simple way to attach just a single SpyCatcher/SpyTag to each IgG. Initially, a preliminary study was performed using glycine-serine linkers between the two pAbBD domains, with lengths of 20 and 50 amino acids. We found that the shorter linker predominantly led to homodimer formation, while the longer linker labeled both heavy chains on a single IgG much more efficiently, with minimal homodimer formation. Therefore, we prepared SpyCatcher and SpyTag constructs with ˜50 amino acids or longer between the two flanking pAbBDs, i.e. pAbBD-(GGS)₇-SC/ST-(GGS)4-pAbBD and pAbBD-(GGS)₇—SC/ST-(GGS)₇-pAbBD (“(GGS)₇” is disclosed as SEQ ID NO: 24 and “(GGS)4” is disclosed as SEQ ID NO: 25). For Cetuximab and Trastuzumab, both designs offered efficient IgG labeling with a single SpyCatcher/SpyTag (>70%). However, for OKT3, the longer fusion protein was needed to achieve comparable labeling efficiency with a single SpyCatcher/SpyTag. In all cases, the fraction of homodimer could be slightly reduced at lower IgG concentrations, but if the concentrations became too low the interaction between SpyCatcher and SpyTag became inefficient when the two IgGs were combined to form BsAbs. Therefore, concentrations of at least 0.1 mg/mL IgG are recommended. Fortunately, the homodimer could be easily removed from IgG labeled with pAbBD-SC/ST-pAbBD by size exclusion chromatography. This led to high purity in IgG labeled with just a single SpyCatcher/SpyTag. Following size-exclusion chromatography and incubation of the single-labeled IgGs at a 1:1 molar ratio, BsAbs are formed with an efficiency of ˜70%. The overall yield of the final BsAb product is 40-50% with ˜95% purity.

It is important to point out that the BsAbs formed from two full-length IgG using the approach described in Scheme 2 are unlikely to be adopted as a therapeutic platform. First, the pAbBD, as well as SpyCathcer/SpyTag, are derived from bacterial proteins and thus could be prone to eliciting an immune response, although it should be noted that bacterial antibody binding domain variants have previously been used in clinical trials. Secondly, all four of the BsAb's C_{H}2-C_{H}3 hinges, two on each antibody, are crosslinked with pAbBDs. The C_{H}2-C_{H}3 junction has been shown to play an important role in IgG recycling through binding to the neonatal Fc receptor (FcR). Therefore, blocking these sites could affect the half-life of these BsAbs in vivo. Full-length IgG have also been shown to bind to Fcγ receptors at the hinge proximal end of C_{H}2, but this site is not modified in this format. Therefore, secondary immune functions such as antibody-dependent cellular cytotoxicity (ADCC) and complement-dependent cytotoxicity (CDC) are likely retained in the IgG×IgG format. Finally, photocrosslinking of IgG with pAbBDs requires exposure to non-damaging 365 nm light for two hours. While this can be performed at milliliter scales, scaling up for clinical use would require further development of crosslinking instrumentation. With these challenges in mind, the methodologies described here are likely best suited for discovery of novel synergistic targets, probing biological systems and uncovering unique signaling pathways. Unique antibody combinations discovered with this approach can then be introduced into other bispecific formats for clinical development. Considering the tetravalent nature of the BsAbs described here, it is envisioned that their behavior will be most predictive of other tetravalent BsAb formats such as those with second targeting domains fused to a native IgG antibody, although this would require further studies to be proven.

### CONCLUSION

In summary, two methods were developed to produce tetravalent, bispecific antibodies from off-the-shelf IgGs in less than a day. Both methods use SpyCatcher and SpyTag, fused to small photoreactive binding domains, to potentiate IgG molecules for BsAb assembly. The Trastuzumab×Cetuximab BsAb binds to each antigen without a significant reduction in affinity compared to each component IgG. The Cetuximab×OKT3 BsAb successfully re-directed and engaged human T cells to facilitate the cytolysis of tumor cells in vitro. Notably, scheme 2 is a rapid, low-cost, and high-yield process that produces highly homogeneous products.

Bispecific antibodies with novel functionalities have largely been developed and tested based on prior knowledge about the potential effects of binding to both molecular targets simultaneously. This approach precludes the discovery of useful and synergistic target pairs, of which the biological mechanisms are yet to be fully understood. The methods presented here enables the use of pre-established, off-the-shelf IgG molecules in BsAb discovery without engineering the IgG architecture using a relatively high-yield, low-cost process. Thus, this represents a powerful new approach to test any combination of antibodies in a BsAb format for unique or synergistic functionalities.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. An adapter comprising a first binding pair member and a pair of photoreactive antibody binding domains (pAbBDs) that are adapted to site-specifically bind and photo-crosslink to an immunoglobulin, wherein said first member of the binding pair member and a second binding pair member comprise two moieties that form a heterodimer, and wherein the pair of pAbBDs and the first binding pair member are connected via linkers.

2. The adapter of claim 1, wherein the two moieties that form a heterodimer are two protein or peptide moieties that form a heterodimer, two moieties that can undergo a click reaction, or a pair of complementary oligonucleotides.

3. The adapter of claim 1, wherein the two moieties form a covalently-linked heterodimer.

4. The adapter of claim 1, wherein each of said pair of pAbBDs in the adapter site-specifically bind and photo-crosslink to the two heavy chains of a single immunoglobulin.

5. The adapter of claim 2, wherein the two protein or peptide moieties that form a heterodimer are selected from the group consisting of SpyCatcher and SpyTag; two complementary halves of a split intein; c-Jun and c-Fos; leucine zippers; split adhesin domains; SnoopCatcher and SnoopTag; S-protein and S-Tag; Streptavidin/Streptactin or variants thereof and Strep-tag or Strep-tag II; calmodulin and calmodulin binding peptide; a leader peptide and a B1 protein pair from a lasso peptide biosynthesis system and a binding pair of a dock-and-lock system. In some embodiments, the binding pair members comprise two moieties that can undergo a click reaction. In some embodiments, the binding pair members comprise a pair of complementary oligonucleotides.

6. The adapter of claim 5, wherein the binding pair members are SpyTag and SpyCatcher.

7. The adapter of claim 1, wherein the immunoglobulin is an IgG molecule.

8. The adapter of claim 1, wherein the adapter is a fusion protein comprising in series a first pAbBD connected via a first linker to the first binding pair member connected via a second linker to a second pAbBD.

9. The adapter of claim 1, wherein the adapter is a fusion protein comprising in series a first pAbBD connected via a first linker to the second pAbBD, and the first binding pair member is connected via a second linker to either the first or the second pAbBD.

10. The adapter of claim 8, wherein the first pAbBD and the second pAbBD site-specifically bind and photo-crosslink to the two heavy chains of a single immunoglobulin.

11. The adapter of claim 8, wherein the first and the second linkers are flexible GS-rich linkers and wherein the flexible GS-rich linkers have from 1 to 8 GGS repeats (SEQ ID NO: 44).

12. The adapter of claim 9, wherein the first linker is between 35 and 100 amino acids in length.

13. A nucleic acid encoding an adapter according to claim 8.

14. An expression vector comprising the nucleic acid of claim 13.

15. A host cell comprising the expression vector of claim 14.

16. A composition comprising:
(a) a first adapter comprising a first binding pair member and a first pair of photoreactive antibody binding domains (pAbBDs) that are adapted to site-specifically bind and photo-crosslink to a first antibody, and wherein the first pair of pAbBDs and the first binding pair member are connected via linkers; and
(b) a second adapter comprising a second binding pair member and a second pair of photoreactive antibody binding domains (pAbBDs) that are adapted to site-specifically bind and photo-crosslink to a second antibody, wherein the second pair of pAbBDs and the second binding pair member are connected via linkers;
wherein the first and second binding pair members comprise two moieties that form a heterodimer.

17. A method of producing a bispecific antibody from a first antibody and a second antibody, comprising:
(a) providing a first adapter comprising a first binding pair member and a first pair of photoreactive antibody binding domains (pAbBDs) that are adapted to site-specifically bind and photo-crosslink to the first antibody, and wherein the first pair of pAbBDs and the first binding pair member are connected via linkers;
(b) providing a second adapter comprising a second binding pair member and a second pair of photoreactive antibody binding domains (pAbBDs) that are adapted to site-specifically bind and photo-crosslink to the second antibody, wherein the second pair of pAbBDs and the second binding pair member are connected via linkers, and wherein the first and second binding pair members comprise two moieties that form a heterodimer;
(c) site-specifically photo-crosslinking each of the first pair of pAbBDs to the first antibody to form a first adapter-antibody conjugate;
(d) site-specifically photo-crosslinking each of the second pair of pAbBDs to the second antibody to form a second adapter-antibody conjugate; and
(e) contacting the first and second adapter-antibody conjugates under conditions where the first and second binding pair members bind to each other to form a bispecific antibody.

18. The method of claim 17, wherein each of the first pair of pAbBDs in the adapter site-specifically bind and photo-crosslink to the each of the two heavy chains in the first antibody, and each of the second pair of pAbBDs in the adapter site-specifically bind and photo-crosslink to the each of the two heavy chains in the second antibody.

19. The method of claim 17, wherein the method further comprises the step of purifying or isolating the bispecific antibody formed in step (e).

20. The method of claim 17, wherein the method further comprises the step of purifying or isolating the first adapter-antibody conjugate after step (c) and/or the step of purifying or isolating the second adapter-antibody conjugate after step (d).


SEQUENCE LISTING (46 sequences)

SEQ ID NO: 1 - protein, 61 aa, Staphylococcus aureus
VDNKFNKEQQNAFYEILHLPNLNEEQRNAFIQSLKDDPSQSANLLAEAKKLNDAQAPKMRM

SEQ ID NO: 2 - protein, 56 aa, Staphylococcus aureus
MTFKLIINGKTLKGEITIEAVDAAEAEKIFKQYANDYGIDGEWTYDDATKTFTVTE

SEQ ID NO: 3 - protein, 47 aa, Homo sapiens
LGGPSVFLFPPKPKDTLMISRTPEVTCVVVDVSHEDPEVKFNWYVDG

SEQ ID NO: 4 - protein, 47 aa, Homo sapiens
VAGPSVFLFPPKPKDTLMISRTPEVTCVVVDVSHEDPEVQFNWYVDG

SEQ ID NO: 5 - protein, 47 aa, Homo sapiens
LGGPSVFLFPPKPKDTLMISRTPEVTCVVVDVSHEDPEVQFKWYVDG

SEQ ID NO: 6 - protein, 47 aa, Homo sapiens
LGGPSVFLFPPKPKDTLMISRTPEVTCVVVDVSQEDPEVQFNWYVDG

SEQ ID NO: 7 - protein, 47 aa, Mus musculus
PEVSSVFIFPPKPKDVLTITLTPKVTCVVVDISKDDPEVQFSWFVDD

SEQ ID NO: 8 - protein, 47 aa, Mus musculus
LGGPSVFIFPPKIKDVLMISLSPIVTCVVVDVSEDDPDVQISWFVNN

SEQ ID NO: 9 - protein, 47 aa, Mus musculus
EGGPSVFIFPPNIKDVLMISLTPKVTCVVVDVSEDDPDVQISWFVNN

SEQ ID NO: 10 - protein, 47 aa, Mus musculus
LGGPSVFIFPPKPKDALMISLTPKVTCVVVDVSEDDPDVHVSWFVDN

SEQ ID NO: 11 - protein, 45 aa, Rattus norvegicus
VSSVFIFPPKTKDVLTITLTPKVTCVVVDISQNDPEVRFSWFIDD

SEQ ID NO: 12 - protein, 45 aa, Rattus norvegicus; identical to SEQ ID NO: 11

SEQ ID NO: 13 - protein, 47 aa, Rattus norvegicus
LGGPSVFIFPPKPKDILLISQNAKVTCVVVDVSEEEPDVQFSWFVNN

SEQ ID NO: 14 - protein, 47 aa, Rattus norvegicus
LGRPSVFIFPPKPKDILMITLTPKVTCVVVDVSEEEPDVQFSWFVDN

SEQ ID NO: 15 - protein, 47 aa, Oryctolagus cuniculus
LGGPSVFIFPPKPKDTLMISRTPEVTCVVVDVSQDDPEVQFTWYINN

SEQ ID NO: 16 - protein, 47 aa, Mesocricetus auratus
LGGPSVFIFPPKPKDVLMISLTPKITCVVVDVSEEEPDVQFNWYVNN

SEQ ID NO: 17 - protein, 80 aa, Artificial Sequence
  Description of Artificial Sequence Synthetic polypeptide
ATHIKFSKRDEDGKELAGATMELRDSSGKTISTWISDGQVKDFYLYPGKYTFVETAAPDGYEVATAITFTVNEQGQVTVN

SEQ ID NO: 18 - protein, 13 aa, Artificial Sequence
  Description of Artificial Sequence Synthetic peptide
AHIVMVDAYKPTK

SEQ ID NO: 19 - protein, 4 aa, Artificial Sequence
  Description of Artificial Sequence Synthetic peptide
  See specification as filed for detailed description of substitutions and preferred embodiments
GGGS

SEQ ID NO: 20 - protein, 5 aa, Artificial Sequence
  Description of Artificial Sequence Synthetic peptide
  See specification as filed for detailed description of substitutions and preferred embodiments
GGGGS

SEQ ID NO: 21 - protein, 5 aa, Artificial Sequence
  Description of Artificial Sequence Synthetic peptide
  MOD_RES (3)..(3): Any amino acid
LPXTG

SEQ ID NO: 22 - protein, 12 aa, Artificial Sequence
  Description of Artificial Sequence Synthetic 12xHis tag
HHHHHHHHHHHH

SEQ ID NO: 23 - protein, 6 aa, Artificial Sequence
  Description of Artificial Sequence Synthetic 6xHis tag
HHHHHH

SEQ ID NO: 24 - protein, 21 aa, Artificial Sequence
  Description of Artificial Sequence Synthetic peptide
GGSGGSGGSGGSGGSGGSGGS

SEQ ID NO: 25 - protein, 12 aa, Artificial Sequence
  Description of Artificial Sequence Synthetic peptide
GGSGGSGGSGGS

SEQ ID NO: 26 - protein, 8 aa, Artificial Sequence
  Description of Artificial Sequence Synthetic peptide
  MOD_RES (3)..(3): Any amino acid
  MOD_RES (8)..(8): Any amino acid
LPXTGGGX

SEQ ID NO: 27 - protein, 35 aa, Homo sapiens
DKSRWQQGNVFSCSVMHEALHNHYTQKSLSLSPGK

SEQ ID NO: 28 - protein, 35 aa, Homo sapiens
DKSRWQQGNIFSCSVMHEALHNRFTQKSLSLSPGK

SEQ ID NO: 29 - protein, 35 aa, Homo sapiens
DKSRWQEGNVFSCSVMHEALHNHYTQKSLSLSLGK

SEQ ID NO: 30 - protein, 35 aa, Mus musculus
QKSNWEAGNTFTCSVLHEGLHNHHTEKSLSHSPGK

SEQ ID NO: 31 - protein, 35 aa, Mus musculus
EKKNWVERNSYSCSVVHEGLHNHHTTKSFSRTPGK

SEQ ID NO: 32 - protein, 35 aa, Mus musculus
KTSKWEKTDSFSCNVRHEGLKNYYLKKTISRSPGL

SEQ ID NO: 33 - protein, 35 aa, Mus musculus
DTDSWLQGEIFTCSVVHEALHNHHTQKNLSRSPEL

SEQ ID NO: 34 - protein, 35 aa, Rattus norvegicus
KKEKWQQGNTFTCSVLHEGLHNHHTEKSLSHSPGK

SEQ ID NO: 35 - protein, 35 aa, Rattus norvegicus
KKETWQQGNTFTCSVLHEGLHNHHTEKSLSHSPGK

SEQ ID NO: 36 - protein, 35 aa, Rattus norvegicus
ERSRWDSRAPFVCSVVHEGLHNHHVEKSISRPPGK

SEQ ID NO: 37 - protein, 35 aa, Rattus norvegicus
DTDSWMRGDIYTCSVVHEALHNHHTQKNLSRSPGK

SEQ ID NO: 38 - protein, 35 aa, Oryctolagus cuniculus
PTSEWQRGDVFTCSVMHEALHNHYTQKSISRSPGK

SEQ ID NO: 39 - protein, 35 aa, Mesocricetus auratus
PKSRWDQGDSFTCSVIHEALHNHHMTKTISRSLGN

SEQ ID NO: 40 - protein, 4 aa, Artificial Sequence
  Description of Artificial Sequence Synthetic peptide
GGGC

SEQ ID NO: 41 - protein, 8 aa, Artificial Sequence
  Description of Artificial Sequence Synthetic peptide
  MOD_RES (3)..(3): Any amino acid
LPXTGGGC

SEQ ID NO: 42 - protein, 24 aa, Artificial Sequence
  Description of Artificial Sequence Synthetic peptide
  SITE (1)..(24): This sequence may encompass 3-8 “Gly Gly Ser” repeating units
GGSGGSGGSGGSGGSGGSGGSGGS

SEQ ID NO: 43 - protein, 90 aa, Artificial Sequence
  Description of Artificial Sequence Synthetic polypeptide
  SITE (1)..(90): This sequence may encompass 4-30 “Gly Gly Ser” repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 44 - protein, 24 aa, Artificial Sequence
  Description of Artificial Sequence Synthetic peptide
  SITE (1)..(24): This sequence may encompass 1-8 “Gly Gly Ser” repeating units
GGSGGSGGSGGSGGSGGSGGSGGS

SEQ ID NO: 45 - protein, 75 aa, Artificial Sequence
  Description of Artificial Sequence Synthetic polypeptide
  SITE (1)..(75): This sequence may encompass 4-25 “Gly Gly Ser” repeating units
GGSGGSGGSGGSGGSGGSGGSGGSGGSGGSGGSGGSGGSGGSGGSGGSGGSGGSGGSGGSGGSGGSGGSGGSGGS

SEQ ID NO: 46 - protein, 60 aa, Artificial Sequence
  Description of Artificial Sequence Synthetic polypeptide
  SITE (1)..(60): This sequence may encompass 4-20 “Gly Gly Ser” repeating units
GGSGGSGGSGGSGGSGGSGGSGGSGGSGGSGGSGGSGGSGGSGGSGGSGGSGGSGGSGGS